(19) United States
(12) Patent Application Publication
(10) Pub. No.: US 2022/0183595 A1
(43) Pub. Date: Jun. 16, 2022

(54) SYSTEMS, DEVICES, AND METHODS FOR ANALYTE SENSOR INSERTION

(71) Applicant: ABBOTT DIABETES CARE INC., Alameda, CA (US)

(72) Inventors: Yi Wang, San Ramon, CA (US); Hyun Cho, Berkeley, CA (US); Benjamin J. Feldman, Berkeley, CA (US); Lam N. Tran, Dublin, CA (US); Zahid Sadik, Tracy, CA (US)

(21) Appl. No.: 17/546,647

(22) Filed: Dec. 9, 2021

Related U.S. Application Data
(60) Provisional application No. 63/123,938, filed on Dec. 10, 2020.

(51) Int. Cl. A61B 5/145; A61B 5/00; A61B 5/151

(52) U.S. Cl. CPC A61B 5/14503; A61B 5/6849; A61B 5/15144; A61B 5/14532; A61B 5/14546

(57) ABSTRACT

Systems, devices and methods are provided for inserting at least a portion of an in vivo analyte sensor for sensing an analyte level in a bodily fluid of a subject. A sensor insertion component may include a small diameter needle disposed at an angle of about 7 to about 10 degrees to a skin normal insertion force vector with a flexible elongate sensor and sharpened tip supported by a U-shaped protector along an intermediate portion. Advancing the needle into the subject along the vector causes stretching of the skin around the needle, allowing entry of the sensor tip into the body. A bump may be provided on a distal portion of the sensor for engagement by the U-shaped protector and transmission of an insertion force to the sensor tip.

### CROSS-REFERENCE TO RELATED APPLICATIONS

This application claims priority to U.S. Provisional Application No. 63/123,938, filed Dec. 10, 2020, which is herein expressly incorporated by reference in its entirety for all purposes.

### FIELD

The subject matter described herein relates generally to systems, devices, and methods for sensor insertion in an in vivo analyte monitoring system.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting a sensor, that senses a user's analyte level in a bodily fluid located in the human body, using an applicator or insertion mechanism, such that the sensor comes into contact with the bodily fluid. The sensor control device may also be configured to transmit analyte data to another device, from which the individual or her health care provider (“HCP”) can review the data and make therapy decisions.

While current sensors can be convenient for users, they are also susceptible to malfunctions due to improper insertion. These malfunctions can be caused by user error, lack of proper training, poor user coordination, overly complicated procedures, and other issues. Some prior art systems, for example, may utilize sharps that are not optimally configured to create an insertion path at the insertion site without creating trauma to surrounding tissue. These challenges and others can lead to improperly inserted or damaged sensors, and consequently, a failure to properly monitor the subject's analyte level.

Certain sharp designs and insertion systems, for example designs in which the sharp has a cross sectional area of about 0.25 mm² or greater, may be associated with a level of sensor insertion trauma causing Early Sensitivity Attenuation (ESA). For a period from a few hours up to a day or two after a new sensor introduced into the tissue, the sensor can suffer a reduction of its sensitivity as known as Early Sensitivity Attenuation or ESA. ESA is mainly due to the tissue reaction to the trauma caused by sensor insertion process. Sensor insertion trauma inhibits newly inserted sensors from accurately measuring and reporting analyte levels for a time after insertion.

Certain sensor insertion systems can use a flex circuit sensor to reduce sensor insertion trauma, and thus, are limited to use with sensors that can be implemented using flex circuits. These designs cannot be used with chip sensors having a substantially greater cross section than flex circuit sensors, which forecloses the use of these sensor insertion systems to insert chip sensors that may be necessary or advantageous for sensing certain analytes or parameters.

Thus, a need exists for sensor insertion devices, systems, and methods that overcome these and other limitations of the prior art, for example, by reducing sensor insertion trauma and associated problems, and enabling insertion of chip sensors for a wider range of applications.

### SUMMARY

Provided herein are example embodiments of systems, devices and methods for the assembly and use of an applicator and a sensor control device of an in vivo analyte monitoring system, and in particular, where flexible elongate sensors are utilized.

In an aspect, a sensor insertion component for use in an applicator of an in vivo analyte sensor may include a sensor module holding a connector coupled with a distal end of a flexible elongate sensor and having at least one surface defining a skin normal insertion force vector, and a sharp module held by the sensor module and configured for motion relative to the sensor module parallel to the skin normal insertion force vector.

The sharp module may include a base configured for the motion relative to the sensor module, for example, sliding motion. The sharp module may further include a U-shaped protector aligned with the skin normal insertion force vector and fixed to the base, having an intermediate portion of the flexible elongate sensor disposed along a length thereof with a distal portion of the flexible elongate sensor extending past a distal end thereof. The intermediate portion of the flexible elongate sensor may be disposed in a channel of the U-shaped protector. The distal portion of the flexible elongate sensor may extend from the U-shaped protector for a length in a range of 0.5 to 4.0 mm, for example. For further example, the U-shaped protector may have a length extending from the base in the range of 1.0 to 10 mm.

The sharp module may further include a sharp fixed to at least one of the base or the U-shaped protector, the sharp having an outer diameter not greater than 0.56 mm and a distal portion extending past a distal end of the flexible elongate sensor at an angle to the skin normal insertion force vector of not less than seven degrees and not greater than ten degrees. The sharp may be, or may include, a solid needle having a diameter not greater than 0.5 mm, for example, a needle having a diameter of about 0.35 mm. In some embodiments, the needle may be an acupuncture-style needle. The distal portion of the sharp may have a length in a range of 1.0 to 5.0 mm, for example.

In other, related aspects, a distal end of the flexible elongate sensor may be sharpened to a point, and may contact or be disposed along a shaft of the sharp. The flexible elongate sensor may further include an attached bump having a traction surface, wherein the traction surface is configured for engagement with the distal end of the U-shaped protector for transmission of an insertion force along the skin normal insertion force vector to the flexible elongate sensor. In further related aspects, the bump may include a sensor chip, optionally encased in a protective membrane, and coupled with the electronics module by a conductor disposed along the flexible elongate sensor. The sensor chip may be, for example, a thermistor for sensing body temperature.

In related aspects, an applicator can be provided to the user in a sterile package with an electronics housing of the sensor control device contained therein. A structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. After assembly, the applicator can be used to position the sensor control device on a human body with a sensor in contact with the wearer's bodily fluid. The embodiments provided herein are improvements to prevent or reduce ESA caused by insertion trauma, and for reducing subject discomfort during insertion. Other improvements and advantages are provided as well. The various configurations of these devices are described in detail by way of the embodiments which are only examples.

In certain embodiments, in vivo analyte sensors are fully integrated with on body electronics (fixedly connected during manufacture), while in other embodiments they are separate but connectable post manufacture (e.g., before, during or after sensor insertion into a body). On body electronics may include an in vivo glucose sensor, electronics, battery, and antenna encased (except for the sensor portion that is for in vivo positioning) in a waterproof housing that includes or is attachable to an adhesive pad.

Systems, devices and methods are provided for inserting at least a portion of an in vivo analyte sensor for sensing an analyte level in a bodily fluid of a subject. A sensor insertion component may include a small diameter needle disposed at an angle of about 8 to about 9 degrees, alternatively about 7 to about 10 degrees, alternatively between about 6 to about 11 degrees, alternatively between about 5 to about 12 degrees, alternatively between about 4 to about 13 degree, alternatively greater than about 7 degrees, alternatively less than about 10 degrees, to a skin normal insertion force vector with a flexible elongate sensor and sharpened tip supported by a U-shaped protector along an intermediate portion. Advancing the needle into the subject along the vector causes stretching of the skin around the needle, allowing entry of the sensor tip into the body. A bump may be provided on a distal portion of the sensor for engagement by the U-shaped protector and transmission of an insertion force to the sensor tip.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device for performing methods as described herein.

FIGS. 2B and 2C are block diagrams depicting example embodiments of sensor control devices.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a subject applying a sensor using an applicator device.

FIG. 3F is a proximal perspective view depicting an example embodiment of a subject with an applied sensor and a used applicator device.

FIG. 4A is a perspective view depicting an example embodiment of a sharp module and sensor module prior to assembly.

FIG. 4B is a perspective view depicting the example embodiment of FIG. 4A after assembly of the sharp and sensor modules.

FIG. 5A is an enlarged side view showing details of a stamped and bladed sharp module with a U-channel for comparison to the hybrid needle sharp module.

FIG. 5B is an enlarged side view showing detailed aspects of a hybrid needle sharp module.

FIG. 6 is an enlarged perspective view of a bent needle attached to a U-shaped protector, for an alternative embodiment of a hybrid needle sharp module.

FIG. 7 is a chart showing absence of ESA in application of a hybrid needle prototypes for glucose monitoring.

FIG. 8 is an enlarged perspective view of a flexible elongate sensor with a chip sensor proximal to a sharpened tip.

FIG. 9A is a side view showing a sharp module assembled to a sensor module having chip sensor, with an enlarged view of the chip sensor.

FIG. 9B is an enlarged view of a sensor with chip sensor, showing additional details.

FIG. 10A show a top schematic view of a hybrid needle assembly with chip sensor.

FIG. 10B shows a side view of a flexible elongate sensor with chip and an enlarged view of a region of the flexible sensor proximal to its tip.

FIG. 11A is an enlarged view similar to FIG. 9B, showing an alternative configuration of a chip sensor coupled with a flexible elongate sensor.

FIG. 11B is an enlarged view similar to FIG. 9B, showing an alternative configuration of a flexible elongate sensor with a non-chip stiffener.

FIGS. 12 and 13 are flow charts illustrating aspects of a method for using sensor insertion components as described herein, or equivalent components, to insert a flexible elongate sensor in or below the skin of a subject.

FIGS. 14A-14D are exemplary diagrams of portions of a support material having a plurality of elongate protectors connected thereto.

FIGS. 15A-15D are exemplary diagrams of portions of a support material having a plurality of elongate protectors and sharps connected thereto.

FIGS. 16A-16D are exemplary diagrams of portions of a support material having a plurality of elongate protectors and sharps connected thereto through an injection molded coupler.

FIGS. 17A-17D are exemplary diagrams of portions of a support material having a mold for an additional injection molded coupler at an end of the sharp.

FIGS. 18A-18F illustrate cross-sectional views depicting an example embodiment of an applicator during a stage of deployment.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein, if any, are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of data supplied by in vivo analyte monitoring systems applied using sensor insertion applicators. Accordingly, many embodiments include in vivo analyte sensors structurally configured so that at least a portion of the sensor is, or can be, positioned in the body of a user to obtain information about at least one analyte of the body. The present disclosure concerns insertion of flex circuit sensors, also called flexible elongate sensors, into human tissue.

For each embodiment of a method disclosed herein, systems and devices capable of performing each of those embodiments are covered within the scope of the present disclosure. For example, embodiments of sensor control devices are disclosed, and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device in connection with any and all of the methods described herein.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, or other terminology. Other devices such as personal computers have also been utilized with or incorporated into in vivo and in vitro monitoring systems.

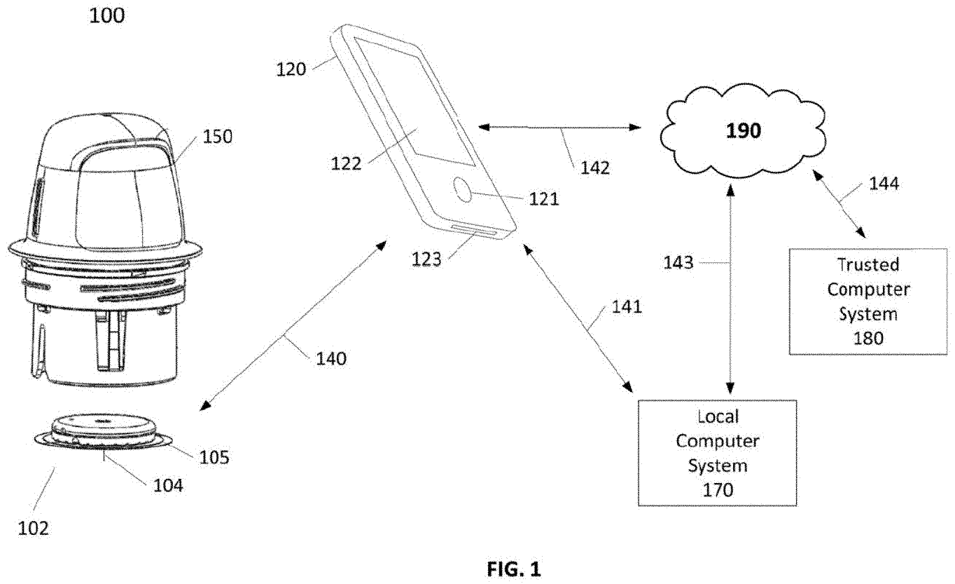

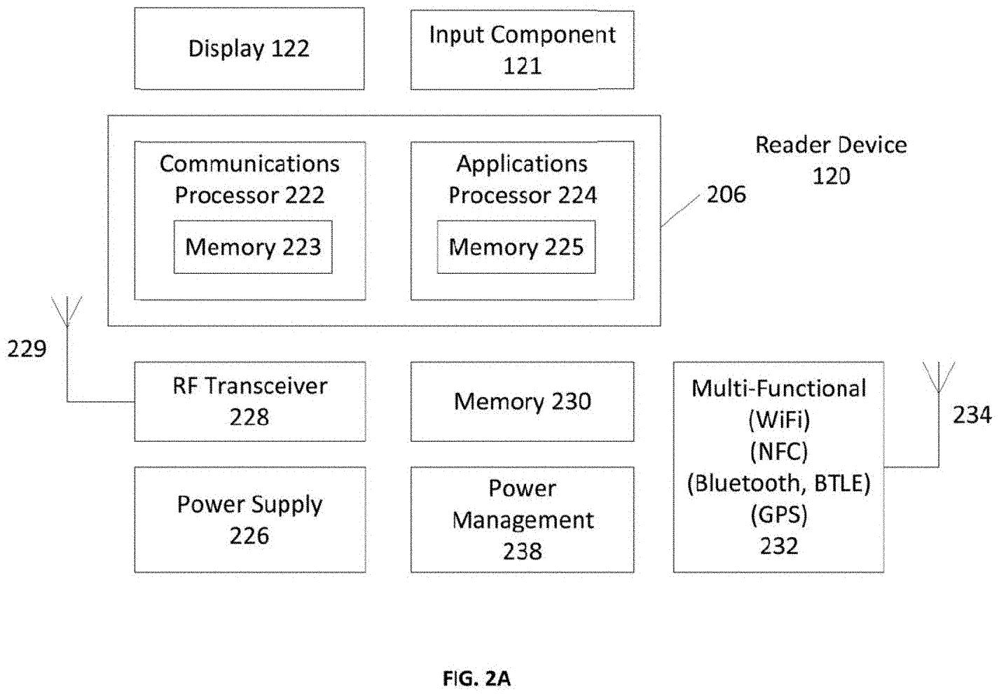

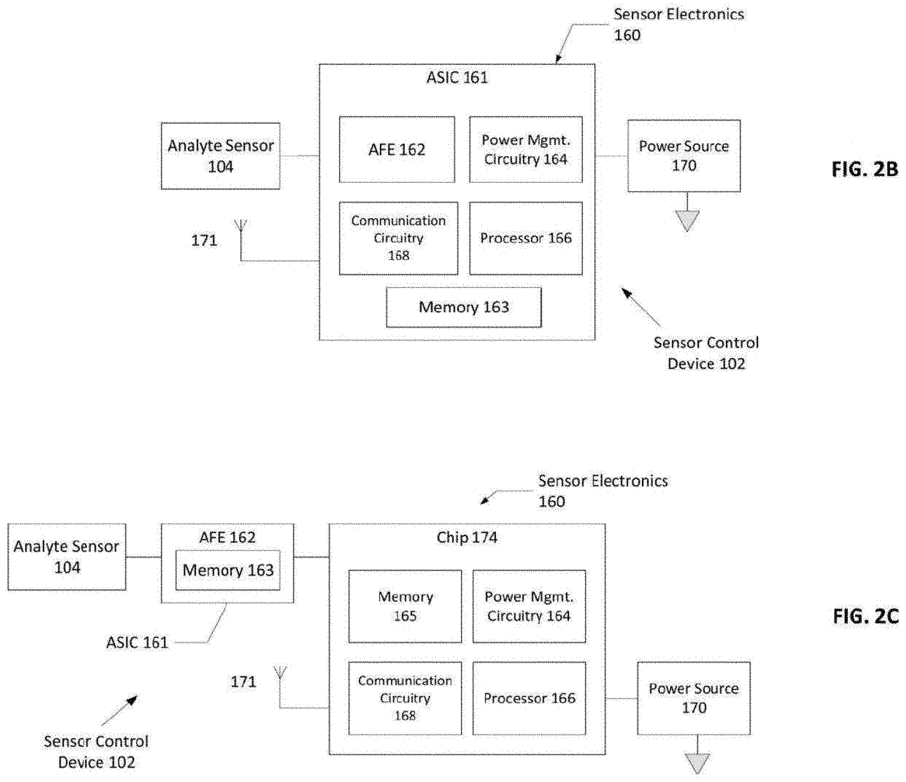

FIG. 1 is a conceptual diagram depicting an example embodiment of an analyte monitoring system 100 that includes a sensor applicator 150, a sensor control device 102, and a reader device 120. The sensor applicator 150 can be used to deliver sensor control device 102 to a monitoring location on a user's skin where a flexible elongate sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

In some embodiments, the sensor control device (e.g., analyte sensor device) may comprise a one-piece architecture that incorporates sterilization techniques specifically designed for a one-piece architecture. The one-piece architecture allows the sensor control device assembly to be shipped to the user in a single, sealed package that does not require any final user assembly steps. Rather, the user need only open one package and subsequently deliver the sensor control device to the target monitoring location. The one-piece system architecture described herein may prove advantageous in eliminating component parts, various fabrication process steps, and user assembly steps. As a result, packaging and waste are reduced, and the potential for user error or contamination to the system is mitigated.

According to some embodiments, a sensor sub-assembly (SSA) can be built and sterilized. The sterilization may be, for example, radiation, such as electron beam (e-beam radiation), but other methods of sterilization may alternatively be used including, but not limited to, gamma ray radiation, X-ray radiation, or any combination thereof. Embodiments of methods of manufacturing an analyte monitoring system using this SSA are now described, as are embodiments of sensor control devices having this SSA and applicators for use therewith. An SSA can be manufactured and then sterilized. During sterilization the SSA can include both an analyte sensor and an insertion sharp. The sterilized SSA can then be assembled to form (e.g., assembled into) a sensor control device, e.g., the sterilized SSA can be placed such that the sensor is in electrical contact with any electronics in a sensor electronics carrier. This sensor control device can then be assembled to form (e.g., assembled into) an applicator (e.g., as a one-piece assembly) where the applicator (also referred to as an analyte sensor inserter) is configured to apply the sensor control device to a user's body. The one-piece assembly can be packaged and/or distributed (e.g., shipped) to a user or health care professional. Additional details regarding sensor control devices can be found in EP 3,897,790, which is hereby expressly incorporated by reference in its entirety for all purposes.

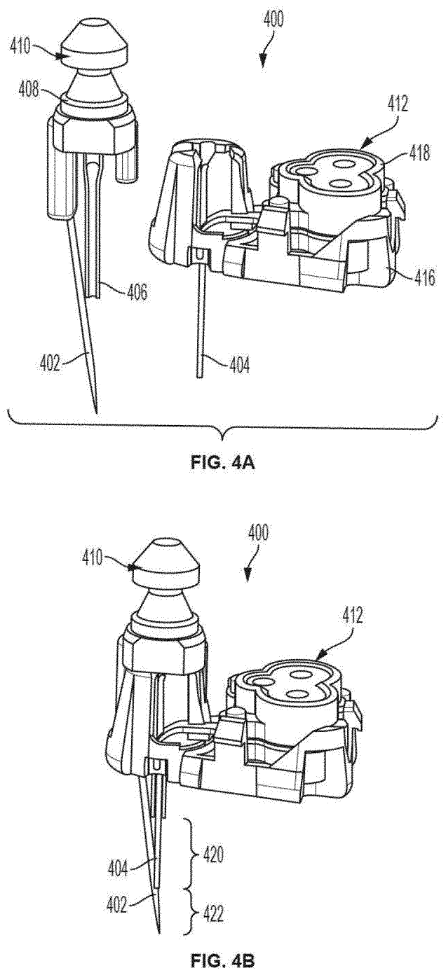

FIG. 2A is a block diagram depicting an example embodiment of a reader device 120 configured as a smartphone. Here, reader device 120 can include a display 122, input component 121, and a processing core 206 including a communications processor 222 coupled with memory 223 and an applications processor 224 coupled with memory 225. The memory 225 may include instructions for performing operations described herein below in connection with FIGS. 4-7. Instructions encoded in the memory 225 may be organized into functional modules that each may be, or may include, a means for performing the function of each functional module. The means may include one or more of the processors 224, 206 and/or 222 coupled to their respective memories, and executing an algorithm based on program instructions stored in the memory. Such algorithm may include a sequence of more detailed operations, as described in connection with the FIGS. 4-7. While the reader device 120 is illustrated with three processors 206, 222 and 224, any useful number of processors may be used or provided in the device 120.

Also included may be separate memory 230, RF transceiver 228 with antenna 229, and power supply 226 with power management module 238. In an alternative, or in addition, the reader device may include a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. The analyte sensor 104 may be configured in a flexible elongate form factor for insertion below the epidermis. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) several different chips.

A memory 163 is also included within ASIC 161 and can be shared by the various functional units present within ASIC 161 or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120, for example, where further processing the resident software application selectively displays relevant portions of the sensor data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

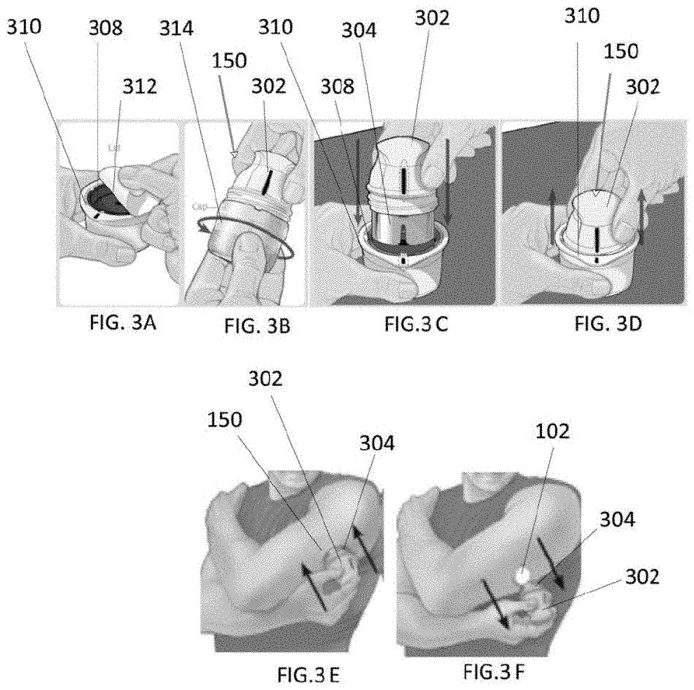

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of a user preparing a container 310, configured here as a tray (although other packages can be used), for an assembly process. The user can accomplish this preparation by removing lid 312 from tray 310 to expose platform 308, for instance by peeling a non-adhered portion of lid 312 away from tray 310 such that adhered portions of lid 312 are removed. Removal of lid 312 can be appropriate in various embodiments so long as platform 308 is adequately exposed within tray 310. Lid 312 can then be placed aside.

FIG. 3B is a side view depicting an example embodiment of a user preparing an applicator device 150 for assembly. Applicator device 150 can be provided in a sterile package sealed by a removable cap 314 for maintaining a sterile environment for the medical device and the sharp housed therein. Preparation of applicator device 150 can include uncoupling housing 302 from cap 314 to expose sheath 304 (FIG. 3C). This can be accomplished by unscrewing (or otherwise uncoupling) cap 314 from housing 302. Cap 314 can then be placed aside.

In some embodiments, the removable cap 314 can be secured to the applicator assembly via complimentary threadings. The end cap may fit with the applicator to create a sterile packaging for interior of the applicator. Therefore, no additional packaging may be required to maintain sterility of the interior of the applicator 150. In some embodiments, the end of the removable end cap may include one or more openings, which can be sealed by a sterile barrier material such as DuPont™ Tyvek®, or other suitable material, to form a seal. Such provision allows for ethylene oxide (ETO) sterilization of the applicator through the seal when closed. In some embodiments, the openings in the removable cap 314 may not be present and the removable cap 314 may be made from a sterile process-permeable material so that the interior of the applicator can be sterilized when the cap 314 is mated to it, but that maintains sterility of the interior of the cap after exposure to the sterility process. In some embodiments, ETO sterilization is compatible with the electronics within the electronics assembly and with the associated adhesive patch, both of which can be releasably retained within the applicator assembly until applied to the user. As shown, the applicator assembly includes a housing including integrally formed grip features and a translating sheath or guide sleeve.

The container 310 and the applicator 150 may be sterilized by different sterilization approaches. For example, a sensor contained in a container 310 may require one type of sterilization process and the contents of an applicator 150—for example, electronics contained within the interior of the applicator—may require another type of sterilization process. The utility of a two-piece separable but combinable system (i.e., the container 310 and the applicator) enables the respective sterilization of the two pieces and sterility maintenance before the two are connected together for use. In other words, separately sealing the container 310 and the applicator 150 facilitates the use of otherwise incompatible sterilization methods for these two components. For example, one type of sterilization that could damage the chemistry of the sensor can be used to sterilize the applicator 150 including the electronics assembly including the adhesive patch. Likewise, another sterilization process that could damage the electronics in the electronics assembly (and/or the adhesive patch used to adhere the electronics assembly to the user's skin) can be used to sterilize the container 310 including the sensor therein. Still other advantages may exist, given different shelf-life attributes for the active (i.e., electronic, chemical, etc.) elements. In some embodiments, all components can be sterilized using the same sterilization technique, such as, but not limited to ETO and e-beam sterilization, etc.

FIG. 3C is a proximal perspective view depicting an example embodiment of a user inserting an applicator device 150 into a tray 310 during an assembly. Initially, the user can insert sheath 304 into platform 308 inside tray 310 after aligning housing orienting feature 1302 (or slot or recess) and tray orienting feature 924 (an abutment or detent). Inserting sheath 304 into platform 308 temporarily unlocks sheath 304 relative to housing 302 and also temporarily unlocks platform 308 relative to tray 310. At this stage, removal of applicator device 150 from tray 310 will result in the same state prior to initial insertion of applicator device 150 into tray 310 (i.e., the process can be reversed or aborted at this point and then repeated without consequence).

Sheath 304 can maintain position within platform 308 with respect to housing 302 while housing 302 is distally advanced, coupling with platform 308 to distally advance platform 308 with respect to tray 310. This step unlocks and collapses platform 308 within tray 310. Sheath 304 can contact and disengage locking features (not shown) within tray 310 that unlock sheath 304 with respect to housing 302 and prevent sheath 304 from moving (relatively) while housing 302 continues to distally advance platform 308. At the end of advancement of housing 302 and platform 308, sheath 304 is permanently unlocked relative to housing 302. A sharp and sensor (not shown) within tray 310 can be coupled with an electronics housing (not shown) within housing 302 at the end of the distal advancement of housing 302. Operation and interaction of the applicator device 150 and tray 310 are further described below.

FIG. 3D is a proximal perspective view depicting an example embodiment of a user removing an applicator device 150 from a tray 310 during an assembly. A user can remove applicator 150 from tray 310 by proximally advancing housing 302 with respect to tray 310 or other motions having the same end effect of uncoupling applicator 150 and tray 310. The applicator device 150 is removed with sensor control device 102 (not shown) fully assembled (sharp, sensor, electronics) therein and positioned for delivery.

FIG. 3E is a proximal perspective view depicting an example embodiment of a subject applying sensor control device 102 using applicator device 150 to a target area of skin, for instance, on an abdomen or other appropriate location. Advancing housing 302 distally collapses sheath 304 within housing 302 and applies the sensor to the target location such that an adhesive layer on the bottom side of sensor control device 102 adheres to the skin. The sharp is automatically retracted into the applicator assembly leaving the sensor in the user and the on-body device sealed from moisture when housing 302 is fully advanced, while the sensor (not shown) is left in position to measure analyte levels. Operation of the applicator 216 when applying the on-body device 222 is designed to provide the user with a sensation that both the insertion and retraction of the sharp 1030 is performed automatically by the internal mechanisms of the applicator 216.

FIG. 3F is a proximal perspective view depicting an example embodiment of a subject with sensor control device 102 in an applied position. The user can then remove applicator 150 from the application site.

System 100, described with respect to FIGS. 3A-3F and elsewhere herein, can provide a reduced or eliminated chance of accidental breakage, permanent deformation, or incorrect assembly of applicator components compared to prior art systems. Since applicator housing 302 directly engages platform 308 while sheath 304 unlocks, rather than indirect engagement via sheath 304, relative angularity between sheath 304 and housing 302 will not result in breakage or permanent deformation of the arms or other components. The potential for relatively high forces (such as in conventional devices) during assembly will be reduced, which in turn improves safety and effectiveness of user assembly. The illustrated sensor control device 102 and applicator device may use useful with the needle apparatus and methods described herein below. Other applicators and sensor control devices may also be useful for use with sensor insertion components and methods as described herein.

Analyte monitoring systems can provide simple and easy-to-use continuous measurement and monitoring of subcutaneous analyte levels, for example, glucose. However, for a period from a few hours up to a day or two after a new sensor is introduced into the tissue, the sensor may suffer a reduction of its sensitivity known as Early Sensitivity Attenuation or “ESA.” ESA is caused at least in part by tissue reaction to the trauma of the sensor insertion process. Thus, minimizing sensor insertion trauma is an important method for reducing or eliminating ESA, enabling a newly inserted sensor to measure and report analyte concentration shortly after insertion.

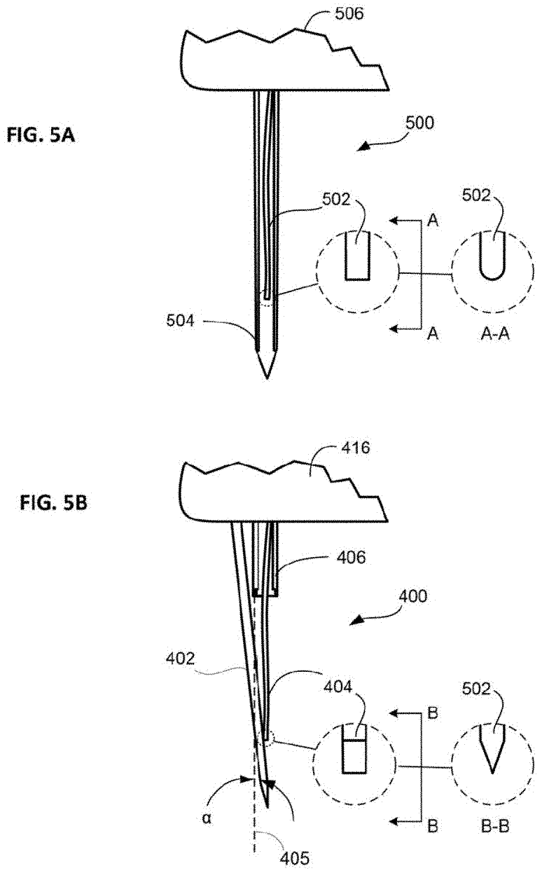

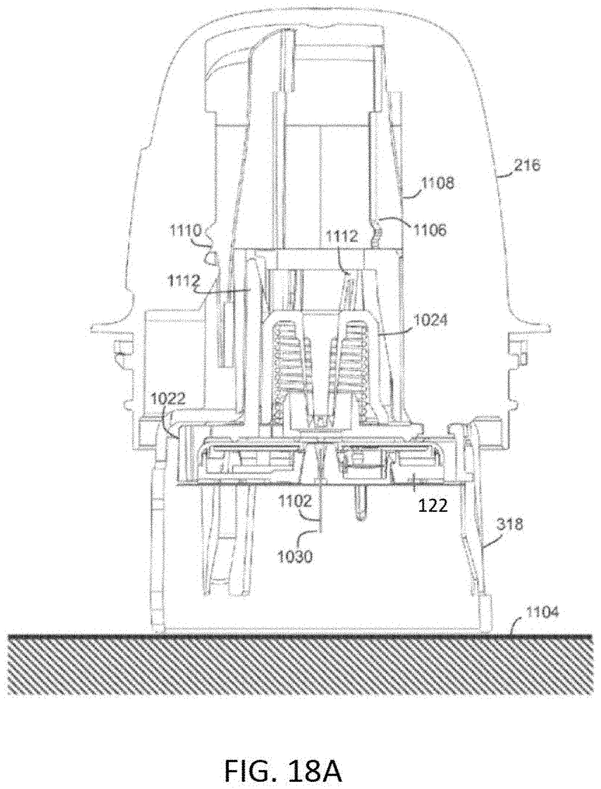

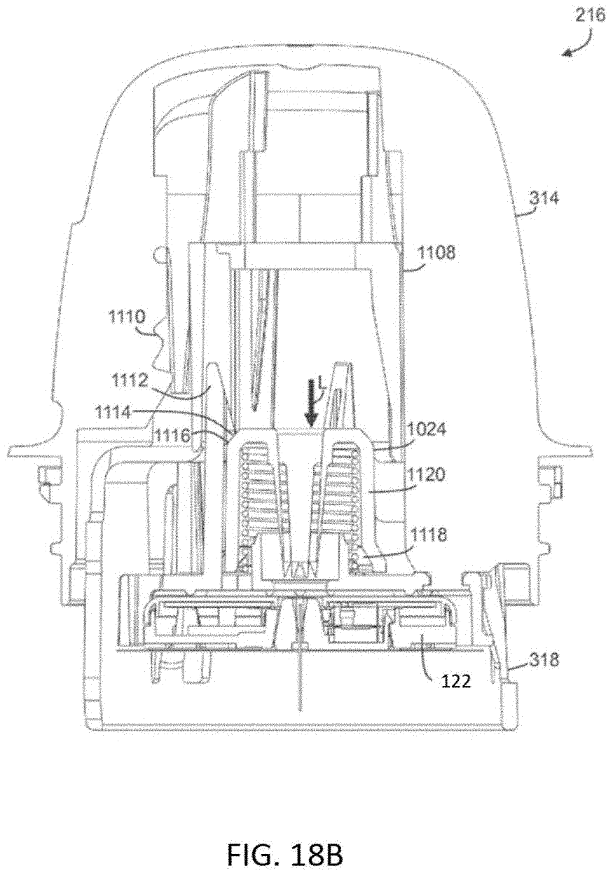

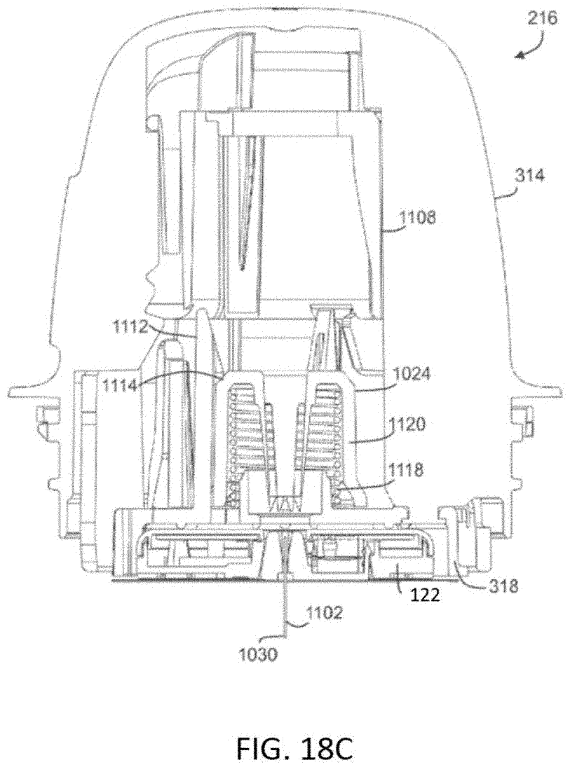

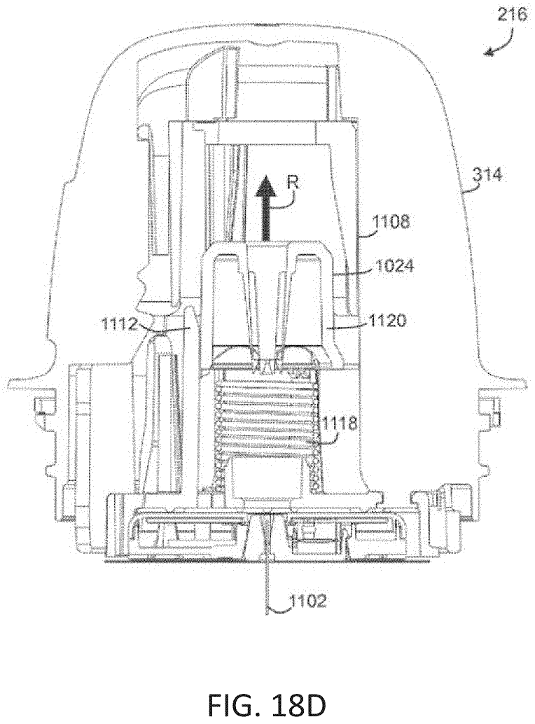

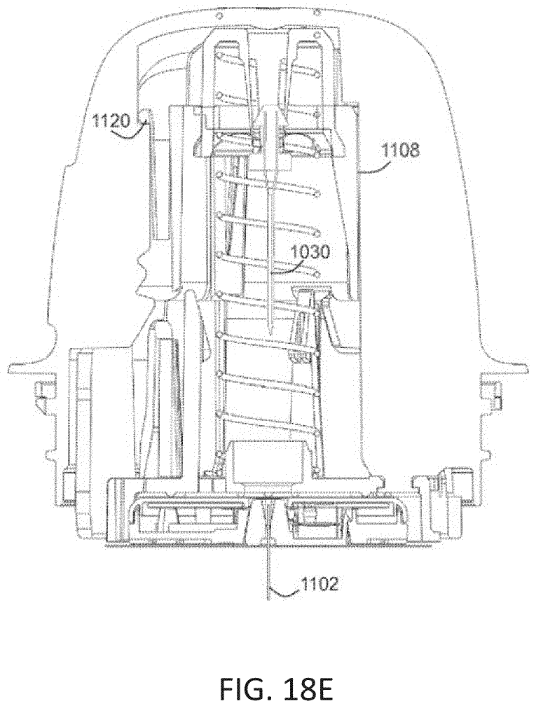

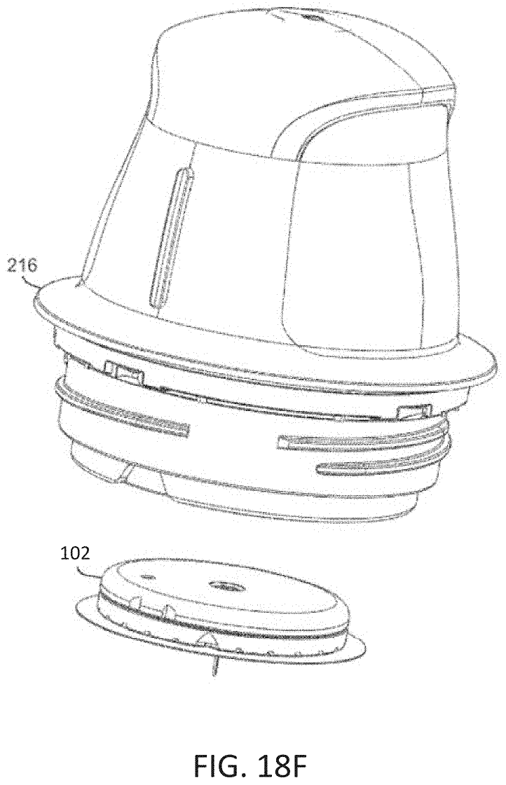

Referring to FIGS. 4A-4B, FIG. 4A shows a sharp module 410, also called a hybrid needle insertion device, separated from a mating sensor control module 412. FIG. 4B shows the sharp module 410 assembled to the sensor control module 412 to provide a sensor module assembly 400 for including in an applicator device as shown and described herein (see, e.g., FIGS. 3A-3F and 18A-18F). FIG. 5B shows, for comparison, an earlier sharp configuration 500 using a U-shaped bladed sharp 504, having a central groove or channel holding a flexible elongate sensor 502 with a rounded tip. A sensor module 506 holds the U-shaped sharp 504 for perpendicular insertion into tissue. The sharp 504 may be a stamp formed U shaped metal sharp with etched cutting edge on its tip. During the insertion process, the etched cutting tip of the sharp 504 makes cuts on the skin while the sensor protected by the groove is pushed into the skin together with metal groove. In the illustrated embodiment, the cross section of the metal groove that goes into the skin is about 0.60×0.53=0.32 mm², which is larger than desired to prevent insertion-trauma caused ESA. Thus, the sharp assembly 500 may result in some degree of ESA, due to the infeasibility of fabricating the relatively complex sharp 504 to be functional, while being small enough to cause no more insertion trauma than the assembly illustrated by FIGS. 4A, 4B and 5B.

Instead of a stamped sharp incorporating a groove, ESA can be reduced or eliminated with a “hybrid needle” that uses a small needle to puncture the skin, in conjunction with a pointed sensor that can be inserted into the piercing made by the needle without a U-shaped groove at the point of insertion. In some embodiments, the needle may be a small acupuncture needle. Insertion trauma may be greatly reduced. For example, the cross-sectional area of a 0.35 mm diameter needle is less than 0.10 mm², less than a third of the illustrated sharp 504. The new hybrid needle insertion process creates much less trauma to the insertion site by making a smaller skin cut and forcing much less volume of sharp material into the tissue. A U-shaped protector may be used to support the sensor strip prior to insertion but does not contact the user's body.

In some embodiments, the needle may be provided with an elongated longitudinal opening or gap in the wall of the sharp, as described in EP 3,766,408, which is hereby expressly incorporated by reference in its entirety for all purposes. In some embodiments, the needle may be fabricated from a sheet of metal, and folded into a substantially “V” or “U” or “C” configuration in cross-section to define the longitudinal recess.

Various technologies can be used to manufacture a folded sheet of metal to form a sharp or needle. For example, etched-sheet metal technology can be used to form the sharp. In this manner, the sharp can be formed having a very sharp edge so that penetration through the skin during insertion is less painful. In other embodiments, a progressive die technology may be utilized to form a complex sheet-metal shape that has a sharp edge. In some embodiments, the sharp can be molded with a plastic cap so that the sharp can be handled during the inserter assembly process. Further, the die cut sharp may be molded with plastic to reinforce the “V,” “U,” or “C” shaped sheet metal configuration. In some embodiments, a “U” shaped cross-section can be provided with having flat, rather than curved walls. The “U” shaped configuration provides the advantage that they can more securely and closely hold the sensor. Also, the “U” shaped configuration provides the advantage that it has a reduced cross-section when compared with a comparable circular cross section. A sharp may have a flat portion, e.g., the bottom of the “U” configuration. A tip may be formed by first distal edges closest to the distal tip and second distal edges between the first distal edges and the substantially parallel side walls. In some embodiments, the first distal edges form an “included tip” angle of about 15 degrees, about 30 degrees, or about 60 degrees. Such angle is symmetrical, that is, equal angles from the longitudinal axis of the sharp. The second distal edges provide a somewhat less acute angle than the first distal edges. In some embodiments, the “lead in” angle may be about 20 degree, about 45 degrees, or about 65 degrees. By having a tip defined by two angles, a first, smaller “included angle” and a second, larger “lead in angle,” allows the tip to meet several objectives. First, the small included angle allows the tip to pierce the skin with less trauma. Second, by broadening out to a larger angle, the overall length of the tip is reduced and strength of the tip is increased.

Referring again to FIGS. 4A-4B, the sharp module 410 may include a small needle 402 having a cross-section of not greater than about 0.25 mm² to puncture the skin and introduce a sharp tipped flexible elongate sensor 404 (also referred to herein as a flexible sensor or sensor) of a sensor control device into the body. The sharp module 410 may further include an elongate U-shaped protector 406 to protect and stabilize an intermediate portion of the flexible sensor 404 during insertion. The U-shaped protector 406 may be formed of any suitable structural material for medical applications, for example, stainless steel, and fixed perpendicularly to the base 408 of the sharp module 410, so that the U-shaped protector is held perpendicular to the skin surface when the sensor module assembly 400 is incorporated into an applicator and applied to a subject. The sharp module 410 may be slidably assembled with the sensor module 416 such that the sharp module 410 can be slid along a vector parallel to the elongate protector 406 (e.g., upwards when the sensor module assembly 400 is oriented as shown in FIG. 4B), to enable retraction of the sharp 402 from the subject's body after the sensor 404 is inserted, while leaving the sensor embedded.

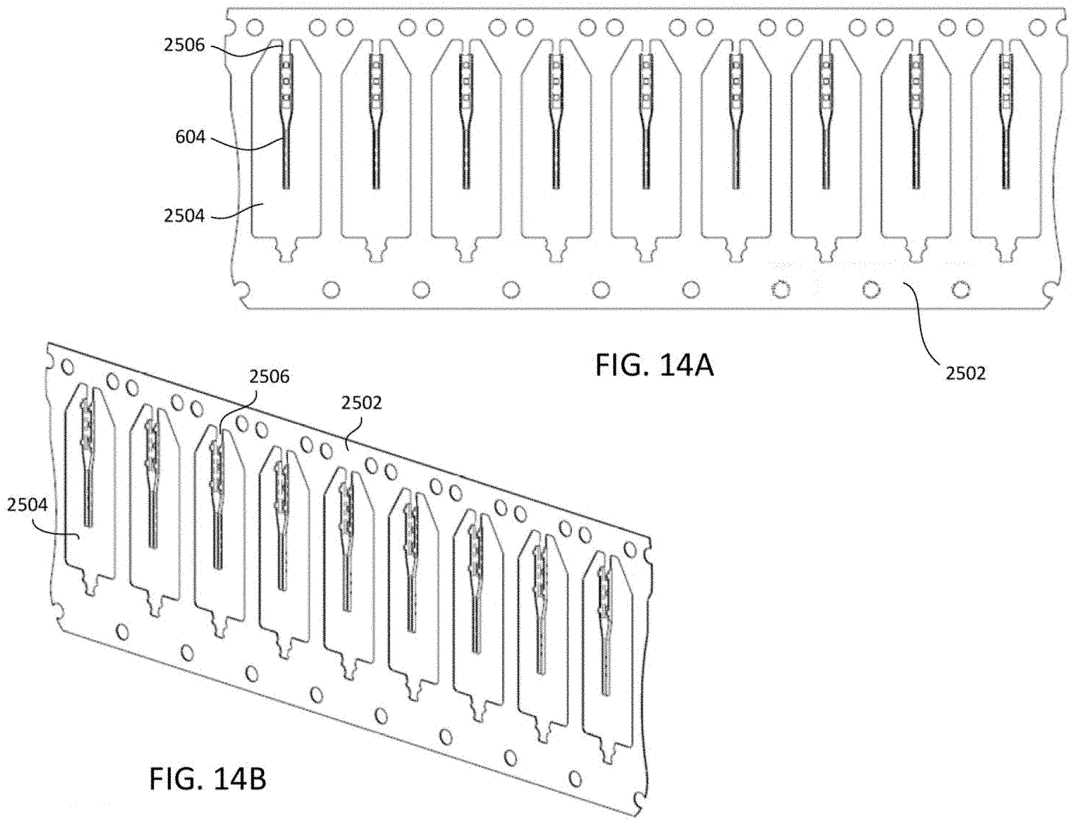

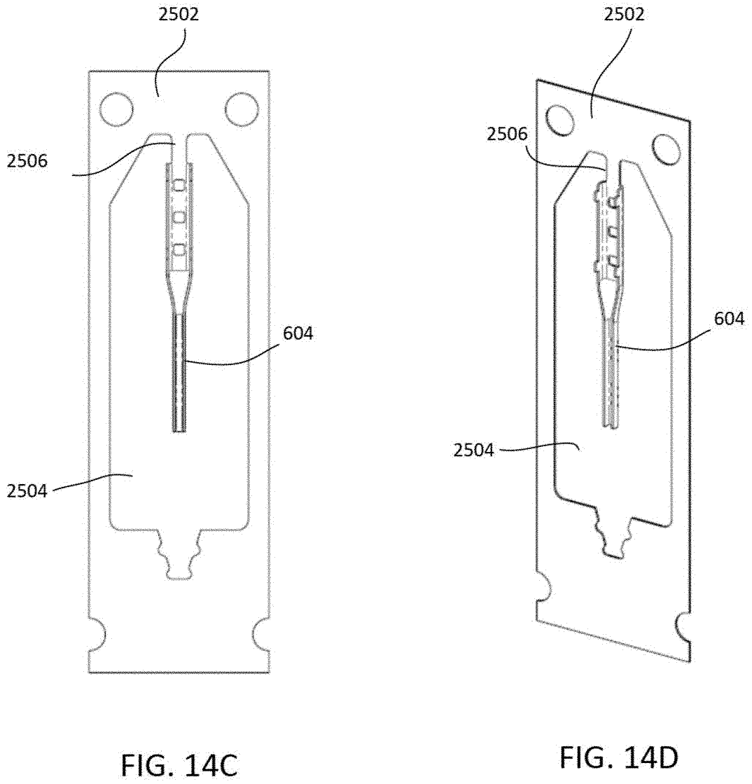

To increase the sensor strength for insertion, the elongate U-shaped protector 406 may be used to protect the flexible elongate sensor. A distal portion 420 of the flexible sensor, for example about 3.0 mm, alternatively about 1.0 mm, alternatively about 2.0 mm, alternatively about 4.0 mm or other suitable length, may be exposed for initial insertion. An additional portion of the flexible sensor body may continue to find its way into the tissue when the hybrid needle retracks from skin. A base component 408 holds the needle 402 in fixed relation to the U-shaped metal groove. The base component 408 may be formed by an injection molding process, wherein a needle may be injection molded into the base component formed of any suitable polymer. This embodiment may support a relatively long needle that punctures skin with an angle, as illustrated. An exemplary method of manufacturing the sharp module 410 is described herein with reference to FIGS. 14-17. Other exemplary manufacturing methods are described in International Publication No. WO 2020/041571, which is hereby expressly incorporated by reference in its entirety for all purposes.

The sensor control module 412 may include the flexible sensor 404, which is coupled with a connector 418 and held in sensor module 416. Connector 418 may be made of silicone rubber that encapsulates compliant carbon impregnated polymer modules that serve as electrical conductive contacts between the sensor and electrical circuitry contacts for the electronics within housing of the on-body unit. The connector can also serve as a moisture barrier for the sensor when assembled in a compressed state after transfer from a container to an applicator and after application to a user's skin. A plurality of seal surfaces can provide a watertight seal for electrical contacts and sensor contacts. One or more hinges can connect two distal and proximal portions of the connector 418.

The sensor module 416 may be configured to accommodate the sharp module 410 so that the flexible sensor 404 fits inside a central groove of the U-shaped protector 406. When assembled into the sensor module 416, the elongate U-shaped protector 406 may be held relative to the sensor module assembly 400 so as to be normal to the skin surface of the subject once integrated into an applicator device and applied to the subject's skin.

The sharp module 410 may include a pointed cylindrical needle 402 held in a fixed orientation to puncture skin with a small angle, not less than about 7 and not greater than about 10 degrees, from the direction of insertion which is perpendicular to the skin surface and indicated by the orientation of the U-shaped protector 406. This small angle stretches the skin opening slightly after the needle punctures the skin to let the flexible sensor 404 having its tip positioned right next to needle 402 into the piercing made by the sharp. A distal portion 422 of the needle 402 extends past the end of the flexible sensor 404 for a distance approximately equal to a desired insertion depth, for example, to about 1.0 mm, alternatively about 1.5 mm, alternatively about 2.0 mm, alternatively about 2.5 mm, alternatively about 3.0 mm, alternatively about 3.5 mm, alternatively about 4.0 mm, alternatively about 4.5 mm, or alternatively about 5.0 mm, past the end of the sensor 404.

The magnitude of the angle a between the sharp 402 and the skin surface normal may be critical to the success of sensor insertion process. FIG. 5B shows the angle a relative to a skin surface normal 405 indicated schematically by a dashed line. If the angle α is greater than about 10 degrees, it may cause the needle to bend and push the sensor off alignment when the needle 402 first engages the skin surface. Conversely, if the angle a is less than about 7 degrees, it may not stretch the skin enough to let the sensor tip into the wound. Because the flexible sensor 404 has essentially the same physical properties as a thin and narrow strip of PET film, it lacks sufficient rigidity to insert itself into the tissue without the piercing and stretching provided by the sharp 402. Additionally, to aid insertion of the flexible sensor into the piercing made by the sharp 405, the distal tip of the flexible sensor 404 may be sharpened as shown in the magnified view of FIG. 5B.

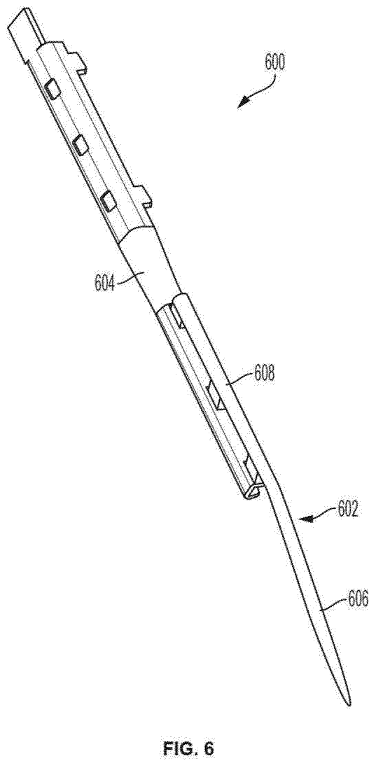

Various other methods may be used to provide a suitably angled small sharp in a sensor application device. For example, as shown in FIG. 6, a proximal portion 608 of a pre-bent needle 602 may be attached (e.g., by spot welding) to an exterior of a U-shaped protector 604 near its distal end. The bend of the sharp 608 delineates its proximal portion 608 from its distal portion 606 and is made at the same angle a as described above. The proximal portion 608 of the sharp 602 is aligned with the U-shaped protector 604 along a shared longitudinal axis. The U-shaped protector 604 may be similar or the same as the U-shaped protector 406, and similarly integrated into the sharp module 410. Advantages of the assembly 600 may include reducing the needle length to improve mechanical rigidity and precision of operation.

Referring generally to FIGS. 4A, 4B, 5B and 6, the needle 402, also called a sharp, may be made of stainless steel or a like resilient material (e.g., material used to manufacture needles, such as acupuncture needles in some embodiments), dimensioned such that the applicator provides for insertion of at least a portion of the sensor 102 into or through human tissue. According to certain embodiments, the sharp may have a cross sectional diameter (width) of from 0.1 mm to 0.5 mm. For example, the sharp may have a diameter of from about 0.1 mm to about 0.3 mm, such as from about 0.15 mm to about 0.25 mm, e.g., about 0.16 mm to about 0.22 mm in diameter. A given sharp may have a constant, i.e., uniform, width along its entire length, or may have a varying, i.e., changing, width along at least a portion of its length, such as the tip portion used to pierce the surface of the skin. In some embodiments, a cross-section of the sharp 402 taken perpendicular to its long axis may not greater than about 0.25 mm². For comparison, based on a sharp having a circular cross-section, a limit of 0.25 mm² is equivalent to a diameter of about 0.56 mm.

For example, a sharp may have a length to insert a sensor to a desired depth, and no more. Insertion depth may be controlled by the length of the sharp, the configuration of the base and/or other applicator components that limit insertion depth. A sharp may have a length between about 1.5 mm and about 25 mm. For example, the sharp may have a length of from about 1 mm to about 3 mm, from about 3 mm to about 5 mm, from about 5 mm to about 7 mm, from about 7 mm to about 9 mm, from about 9 mm to about 11 mm, from about 11 mm to about 13 mm, from about 13 mm to about 15 mm, from about 15 mm to about 17 mm, from about 17 mm to about 19 mm, from about 19 mm to about 21 mm, from about 21 mm to about 23 mm, from about 23 mm to about 25 mm, or a length greater than about 25 mm. It will be appreciated that while a sharp may have a length up to about 25 mm, in certain embodiments the full length of the sharp is not inserted into the subject because it would extend beyond a desired depth. Non-inserted sharp length may provide for handling and manipulation of the sharp in an applicator set. Therefore, while a sharp may have a length up to about 25 mm, the insertion depth of the sharp in the skin on a subject in those certain embodiments may be limited to a desired depth, e.g., about 1.5 mm to about 4 mm, depending on the skin location, as described in greater detail below. For example, in some embodiments disclosed herein, the sharp can be configured to extend into (or even fully through) subcutaneous tissue (e.g., about 3 mm to about 10 mm beneath the surface of the skin depending on the location of the skin on the body). Additionally, in some example embodiments, the sharps described herein can include hollow or partially hollow insertion needles, having an internal space or lumen. In other embodiments, however, the sharps described herein can include solid insertion needles, which do not have an internal space and/or lumen. Furthermore, a sharp of the subject applicator sets can also be bladed or non-bladed.

The dimensions (e.g., the length) of the sensor may be selected according to the body site of the subject in which the sensor is to be inserted, as the depth and thickness of the epidermis and dermis exhibit a degree of variability depending on skin location. For example, the epidermis is only about 0.05 mm thick on the eyelids, but about 1.5 mm thick on the palms and the soles of the feet. The dermis is the thickest of the three layers of skin and ranges from about 1.5 mm to 4 mm thick, depending on the skin location. Methods may include determining an insertion site on a body of a user and determining the depth of the layer at the site, and selecting the appropriately-sized applicator set for the site.

In some embodiments, the sensor 404 is an elongate sensor having a longest dimension (or “length”) of from 1.0 mm to 10 mm. The length of the sensor that is inserted, in the embodiments in which only a portion of a sensor is inserted, ranges from about 0.5 mm to about 7 mm, such as from about 4 mm to about 6 mm, e.g., about 5 or about 6 mm. The flexible elongate sensor may have an aspect ratio of length to width (diameter) of not less than 3:1, for example, 10:1, 20:1, etc. The inserted portion of a sensor has sensing chemistry.

Those of skill in the art will understand that embodiments of the sensor control device can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

The use of a small sharp at the acute angle of about 7 to about 10 degrees may cause, upon normal insertion, a lateral force on the needle towards the sensor tip. This lateral force is a function of skin toughness or resistance. For tough skin, the lateral force may be great enough to bend or displace the tip of the sharp out of alignment with the sensor tip, resulting in a failed insertion. Using a configuration as illustrated in FIGS. 4A-4B in an initial study with multiple insertions on 13 subjects, the insertion success rate was above 96%, wherein most failures were due to prototype applicator misfiles. A sharp configuration as shown in FIG. 6 should reduce sensor failures due to lateral forces from tough skin, as should better controlled manufacturing during regular production. Thus, insertion failure should be much less than experienced with the prototypes.

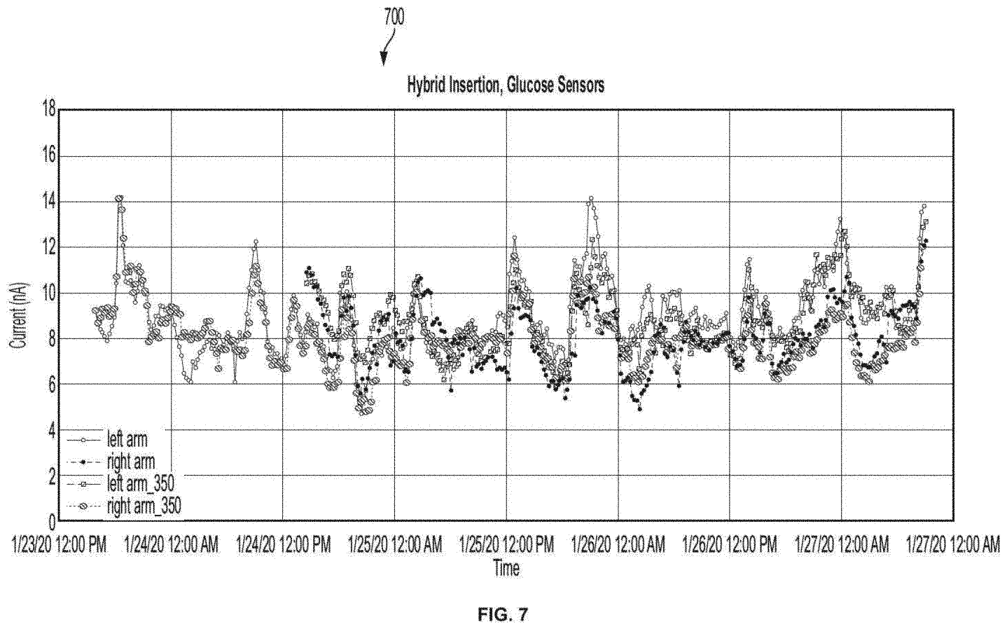

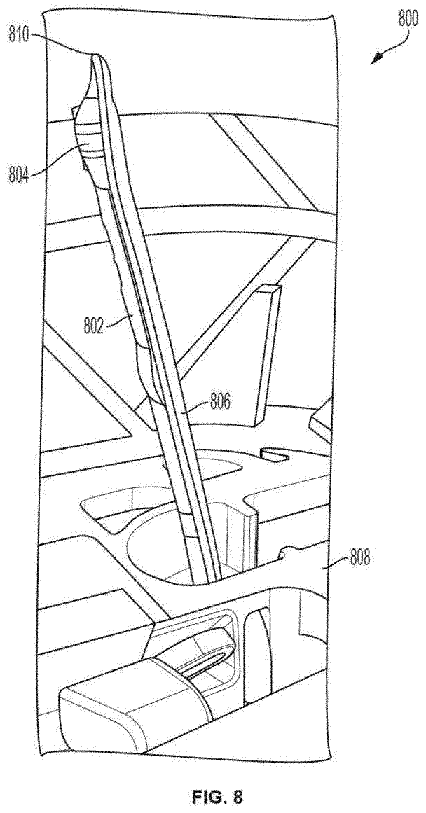

Nonetheless, the prototypes demonstrated a significant reduction of ESA. The chart 700 of FIG. 7 illustrates an example of glucose data from four sensors on the same subject applied using hybrid needle insertion devices. Two sensors (labeled with suffix “_350”) were inserted about 24 hours after insertion of the first two. Sensor data coheres well throughout the test period. Little or no indication of systematic error is apparent. No indication of insertion trauma induced ESA is apparent.

FIGS. 18A-18F illustrate example details of embodiments of the internal device mechanics of “firing” the applicator 216 to apply sensor control device 102 to a user and including retracting sharp 1030 safely back into used applicator 216. All together, these drawings represent an example sequence of driving sharp 1030 (supporting a sensor coupled with sensor control device 102) into the skin of a user, withdrawing the sharp while leaving the sensor behind in operative contact with interstitial fluid of the user, and adhering the sensor control device to the skin of the user with an adhesive. Modification of such activity for use with the alternative applicator assembly embodiments and components can be appreciated in reference to the same by those with skill in the art. Moreover, applicator 216 may be a sensor applicator having one-piece architecture or a two-piece architecture as disclosed herein.

Turning now to FIG. 18A, a sensor 1102 is supported within sharp 1030, just above the skin 1104 of the user. Rails 1106 (optionally three of them) of an upper guide section 1108 may be provided to control applicator 216 motion relative to sheath 318. The sheath 318 is held by detent features 1110 within the applicator 216 such that appropriate downward force along the longitudinal axis of the applicator 216 will cause the resistance provided by the detent features 1110 to be overcome so that sharp 1030 and sensor control device 102 can translate along the longitudinal axis into (and onto) skin 1104 of the user. In addition, catch arms 1112 of sensor carrier 1022 engage the sharp retraction assembly 1024 to maintain the sharp 1030 in a position relative to the sensor control device 102.

In FIG. 18B, user force is applied to overcome or override detent features 1110 and sheath 318 collapses into housing 314 driving the sensor control device 102 (with associated parts) to translate down as indicated by the arrow L along the longitudinal axis. An inner diameter of the upper guide section 1108 of the sheath 318 constrains the position of carrier arms 1112 through the full stroke of the sensor/sharp insertion process. The retention of the stop surfaces 1114 of carrier arms 1112 against the complimentary faces 1116 of the sharp retraction assembly 1024 maintains the position of the members with return spring 1118 fully energized.

In FIG. 18C, sensor 1102 and sharp 1030 have reached full insertion depth. In so doing, the carrier arms 1112 clear the upper guide section 1108 inner diameter. Then, the compressed force of the coil return spring 1118 drives angled stop surfaces 1114 radially outward, releasing force to drive the sharp carrier 1102 of the sharp retraction assembly 1024 to pull the (slotted or otherwise configured) sharp 1030 out of the user and off of the sensor 1102 as indicated by the arrow R in FIG. 18D.

With the sharp 1030 fully retracted as shown in FIG. 18E, the upper guide section 1108 of the sheath 318 is set with a final locking feature 1120. As shown in FIG. 18F, the spent applicator assembly 216 is removed from the insertion site, leaving behind the sensor control device 102, and with the sharp 1030 secured safely inside the applicator assembly 216. The spent applicator assembly 216 is now ready for disposal.

In some embodiments, the retractor withdraws the sharp upon actuation by the user. In such cases, the user actuates the retractor when it is desired to withdraw the sharp. For example, the retractor may include a release switch. Upon activation of the release switch, the drive assembly, e.g., the spring or other driver, retracts the sharp from the skin. In other embodiments, the retractor and the actuator comprise common components. After activating the actuator to advance the sharp and the analyte sensor, the user releases the actuator, which allows the drive assembly to withdraw the sharp from the skin.

In some embodiments, the retractor withdraws the sharp without further user interaction after actuation of insertion. For example, the inserter may include features or components which automatically retract the sharp upon advancement of the sharp and support structure by a predetermined amount. Inserter devices, in which no further action by the user is required to initiate withdrawal of the sharp after insertion, may be referred to herein as having “automatic” withdrawal of the sharp.

Operation of the applicator 216 when applying the sensor control device 102 is designed to provide the user with a sensation that both the insertion and retraction of the sharp 1030 is performed automatically by the internal mechanisms of the applicator 216. In other words, the present invention avoids the user experiencing the sensation that he is manually driving the sharp 1030 into his skin. Thus, once the user applies sufficient force to overcome the resistance from the detent features of the applicator 216, the resulting actions of the applicator 216 are perceived to be an automated response to the applicator being “triggered.” The user does not perceive that he is supplying additional force to drive the sharp 1030 to pierce his skin despite that all the driving force is provided by the user and no additional biasing/driving means are used to insert the sharp 1030. As detailed above in FIG. 18C, the retraction of the sharp 1030 is automated by the coil return spring 1118 of the applicator 216.

With respect to any of the applicator embodiments described herein, as well as any of the components thereof, including but not limited to the sharp, sharp module and sensor module embodiments, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

Additionally, with respect to any of the applicator embodiments described herein, those of skill in the art will understand that an analyte sensor, as well as one or more structural components coupled thereto, including but not limited to one or more spring-mechanisms, can be disposed within the applicator in an off-center position relative to one or more axes of the applicator. In some applicator embodiments, for example, an analyte sensor and a spring mechanism can be disposed in a first off-center position relative to an axis of the applicator on a first side of the applicator, and the sensor electronics can be disposed in a second off-center position relative to the axis of the applicator on a second side of the applicator. In other applicator embodiments, the analyte sensor, spring mechanism, and sensor electronics can be disposed in an off-center position relative to an axis of the applicator on the same side. Those of skill in the art will appreciate that other permutations and configurations in which any or all of the analyte sensor, spring mechanism, sensor electronics, and other components of the applicator are disposed in a centered or off-centered position relative to one or more axes of the applicator are possible and fully within the scope of the present disclosure.

A number of deflectable structures are described herein, including but not limited to deflectable detent snaps 1402, deflectable locking arms 1412, sharp carrier lock arms 1524, sharp retention arms 1618, and module snaps 2202. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened). In many instances these structures are configured as arms with detents, or snaps, but other structures or configurations can be used that retain the same characteristics of deflectability and ability to return to a resting position, including but not limited to a leg, a clip, a catch, an abutment on a deflectable member, and the like.

In certain embodiments, the sensor positioning process is automatic in that a user need only activate the device, e.g., actuate a button, lever, contact with a skin surface, or the like, to initiate the sensor positioning process, which process then proceeds to completion without any further user intervention.

Additional details of suitable devices, systems, methods, components and the operation thereof along with related features are set forth in International Publication No. WO 2018/136898 to Rao et. Al., International Publication No. WO 2019/236850 to Thomas et. Al., International Publication No. WO 2019/236859 to Thomas et. Al., International Publication No. WO 2019/236876 to Thomas et. Al., and U.S. Patent Publication No. 2020/0196919, filed Jun. 6, 2019, each of which is incorporated by reference in its entirety herein. Further details regarding embodiments of applicators, their components, and variants thereof, are described in U.S. Patent Publication Nos. 2019/0282137, 2021/0219887, 2019/0347086, 2013/0150691, 2016/0331283, and 2018/0235520, all of which are incorporated by reference herein in their entireties and for all purposes. Further details regarding embodiments of sharp modules, sharps, their components, and variants thereof, are described in U.S. Patent Publication No. 2014/0171771, which is incorporated by reference herein in its entirety and for all purposes.

Additionally, with respect to any of the applicator embodiments described herein, sensor module assembly 400, which includes the elongate protector 604 and the sharp 402 oriented at an angle with respect to the longitudinal axis of the elongate protector 604, may be used in conjunction with the applicator 206 described with reference to FIGS. 18A-18F.

The embodiments as discussed above may be useful for inserting a lab-on-chip type sensor made from MEMS or wafer technology into tissue for continuous sensing. A small, off-axis needle as described for the hybrid needle embodiments is adapted for inserting a sensor assembly 800. The sensor assembly 800 may include a sharp-tipped, flexible elongate sensor 802 with a chip sensor 804 attached near the tip 810 of the flexible sensor. The chip sensor 804 may be relatively small, for example, about 0.4×0.2×0.2 mm. A separate conductor 806 connects the chip sensor to an electronic circuitry in the supporting sensor module 808. The chip sensor 804 may be used to monitor a different parameter than the flexible sensor 802. For example, the chip sensor 804 may be, or may include, a thermistor for measuring the sensor site temperature.

As in the hybrid needle described above, a small needle may be used to puncture the skin and introduce a sharp tipped sensor carrier 802 with attached chip sensor 804 into the piercing made by the needle. A U-shaped metal protector as described above may facilitate insertion by pushing on the mounted sensor chip 804 with its leading edge during the insertion process, while supporting and protecting the flexible sensor. Thus, a chip sensor and flexible sensor for continuous analyte monitoring can be introduced into the sensor site with minimum insertion trauma and reduction of Early Sensitivity Attenuation (ESA), as demonstrated for the hybrid needle device.

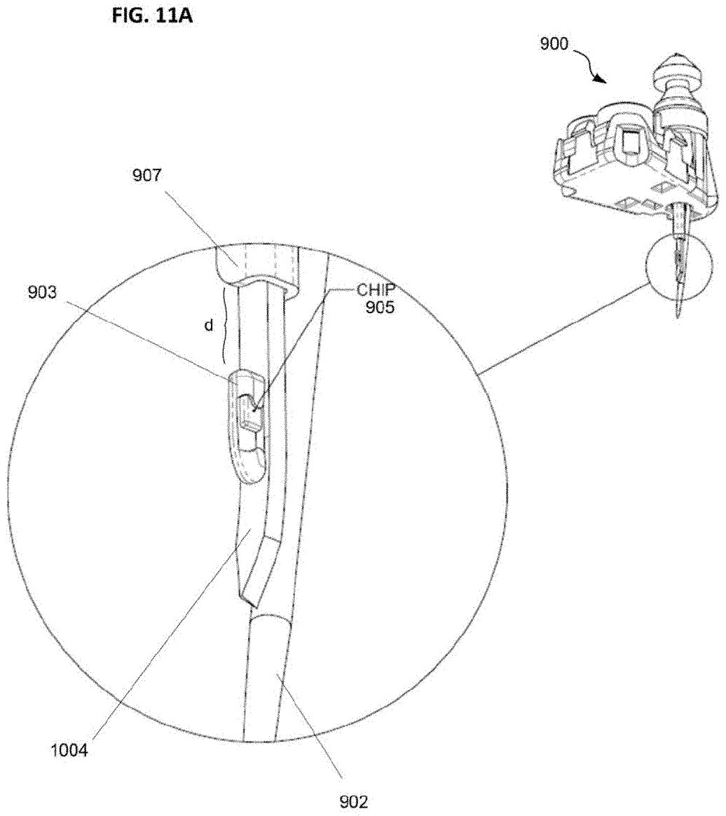

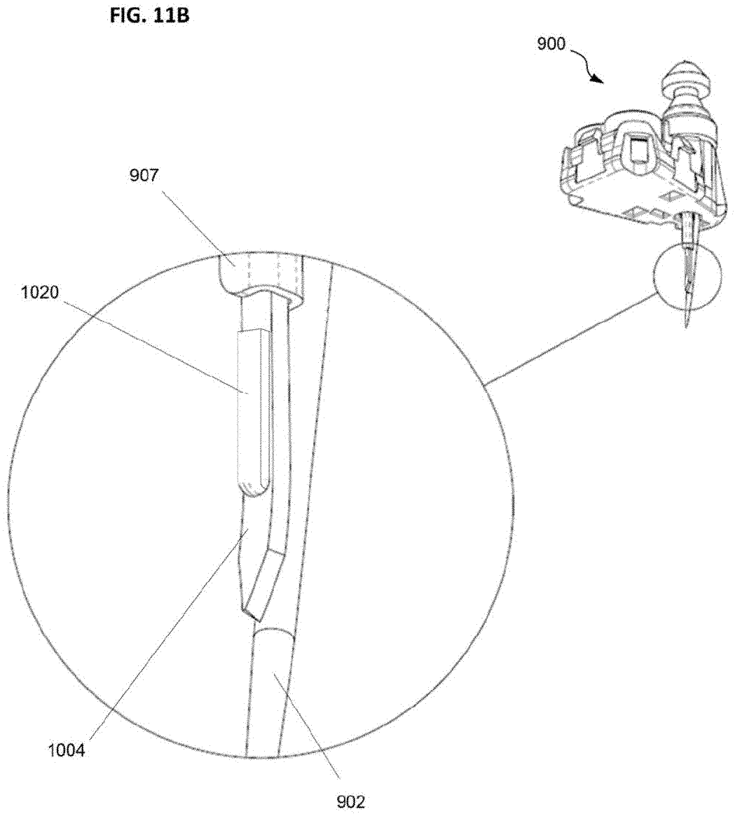

The chip sensor 804 may be, or may include, a sensor made with MEMS or wafer technology. In embodiments, the chip sensor 804 is thicker than the flexible sensor 802 due to the former's substrate and layering construction. Thus, a chip sensor assembly 800 as shown in FIG. 8 would be very difficult to insert with guiding sharps as shown in FIGS. 5A or 11A, or using a hollow needle that requires the sensor assembly to fit inside the sharp's hollow interior. A sharp would need to be much larger than sharps currently in use to insert a laminated chip sensor assembly 800. Accordingly, pain and trauma would be caused by sensor insertion of a composite chip and flexible sensor. In contrast, a hybrid needle assembly can introduce a lab-on-chip type composite sensor 800 into tissue subcutaneously for continuous measurement, with minimal trauma.

Advantageously, the tip-mounted chip 804 can reduce the precision alignment required for sensor insertion in a hybrid needle device. As noted above, a hybrid needle sensor insertion relies on a precise alignment between sensor and needle for successful insertion of the flexible sensor. The force pushing the flexible sensor into skin is applied at the base of the sensor where it is bent at approximately 90° for connection to the connector. Thus, the insertion force is applied relatively distal from the sensor tip and transferred through the thin flexible sensor body to push the sensor tip into skin. Although the large portion of the flexible sensor body is protected by the U-shape metal protector, a few millimeters proximal to the sensor tip is exposed at the end of U-shape metal protector. A slight misalignment at the sensor tip may therefore cause the flexible sensor tip to bend during the insertion, causing insertion failure.

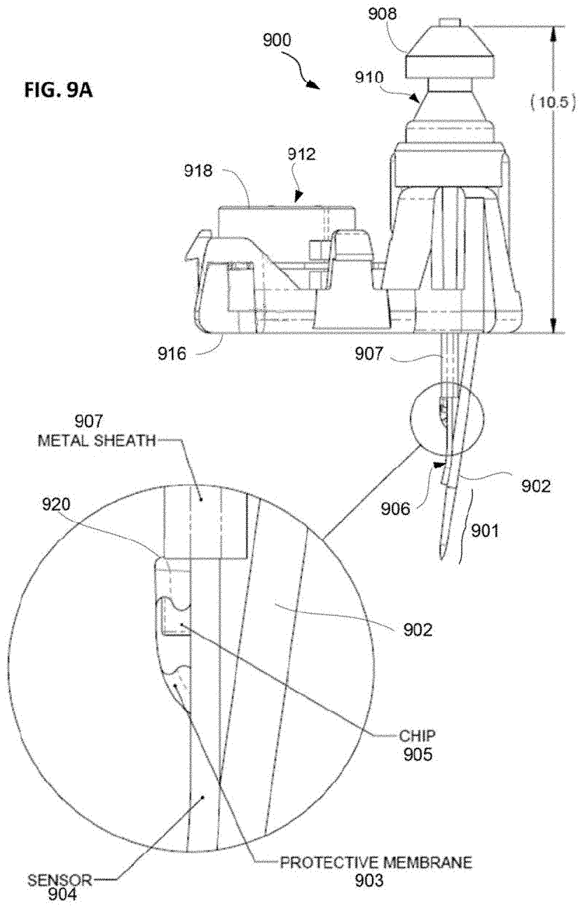

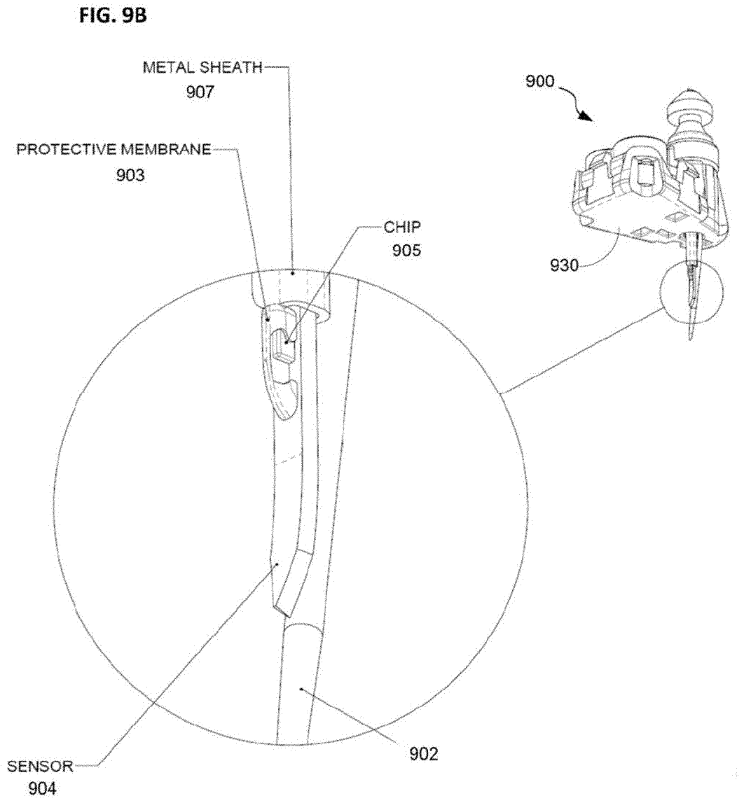

In comparison, adding the small inflexible chip 804 proximal to the flexible sensor tip on a side a surface aligned with the end of U-shape metal protector 907, as shown in FIG. 9A, enables use of the protector 907 to push the chip 905 and sensor tip 905 into a piercing made by the needle 902. A distal portion 901 of the needle 902 extends past the end of the sensor assembly 906 for a distance equal to or not less than a desired insertion depth, inclined at an angle of between about 7 to about 10 degrees, alternatively between about 6 to about 11 degrees, alternatively between about 5 to about 12 degrees, alternatively between about 4 to about 13 degree, alternatively greater than about 7 degrees, alternatively less than about 10 degrees, to the skin normal insertion vector with which the protector 907 is aligned. As shown in FIG. 9A, the needle 902 is tapered to a point along at least a portion of its distal portion 901.

The chip 905 may be encased in a protective membrane 903, for example, a bio-compatible polymer membrane, for protection from mechanical or electrical damage. In the illustrated configuration, the mounted inflexible chip 905 increases sensor tip rigidity for easy insertion while enabling application of the insertion force near the distal end (tip) of the sensor 904 on the mounted chip by the U-shape metal protector instead of the far away base of sensor. Thus, the combination of the chip with the protector ensures the sensor tip is pushed into the hole created by a small needle before the needle and U-shape metal protector are retracted.

FIGS. 9A-9B show aspects of a sensor module assembly 900 using a flexible sensor carrier 906 with a chip 905 mounted proximal to a tip of the sensor 904, enabling an insertion force to be applied to the sensor tip by the U-shaped protector 907 during an insertion process. Dimensions are shown in millimeters. The needle 902 and protector 907 may be formed from any suitable medical material, for example stainless steel, and fixed to a slideable base 908, making up a retractable hybrid needle module 910. The module 910 can be retracted out of the sensor module 916 after insertion of the sensor 904, thereby retracting the needle 902 from the subject's body. Likewise, prior to application, the sharp module 910 is held in the sensor module and during application is pushed towards the subject's body to the position shown in FIG. 9A. A lower surface 930 (FIG. 9B) of the sensor module 916 contacts or parallels the subject's skin during application, defining a skin normal (i.e., perpendicular to subject's skin) insertion force vector collinear with the U-shaped protector 907, to which the needle 902 is inclined at an angle in the range of about 7 to about 10 degrees, alternatively between about 6 to about 11 degrees, alternatively between about 5 to about 12 degrees, alternatively between about 4 to about 13 degree, alternatively greater than about 7 degrees, alternatively less than about 10 degrees, as described herein above. Any suitable surface of the sensor module may be used to define the skin normal insertion force vector. Such an alignment surface is not limited to the lower surface 930 or to a surface parallel to the skin surface, and may include any surface of the sensor module used for aligning to an applicator of a sensor control device in which the sensor insertion module 900 is included. The sensor module 916 holds connector 918 and the sensor assembly 906.

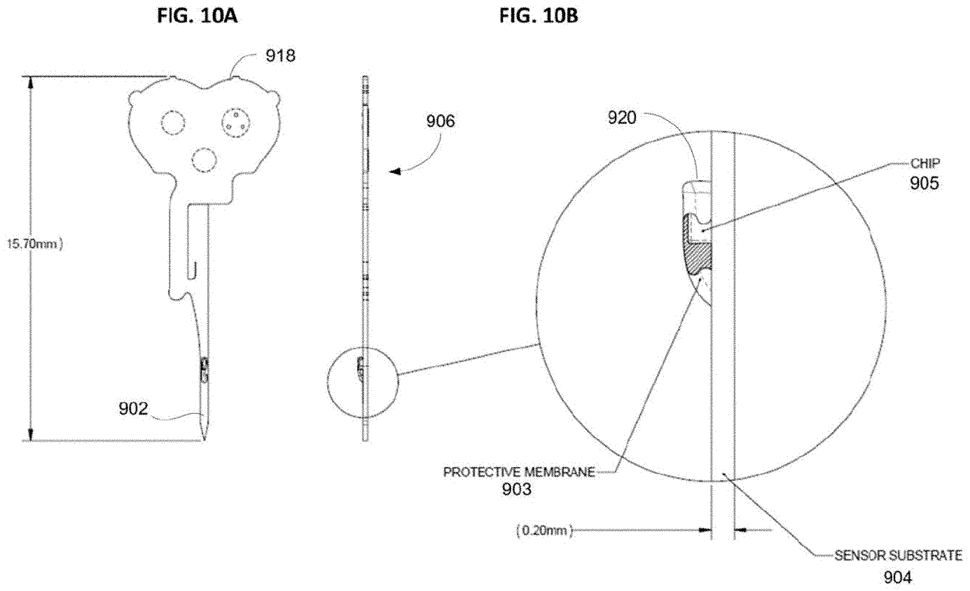

FIG. 9B shows another enlarged view of the tip configuration for the sensor 904, in the sensor module assembly 900. Alternative views are provided by FIGS. 10A and 10B. The chip 905 and protective membrane 903 are positioned near the sharpened tip of the sensor assembly 906, for example, within a range of about zero to about four millimeters, such as, for further example, in a range of about 0 to about 0.1 mm, about 0.1 mm to about 1 mm, about 0.2 to about 2 mm, about 0.3 to about 3 mm, about 0.4 to about 4 mm, about 1 to about 2 mm, or about 1 to about 3 mm. One of ordinary skill may select a suitable setback from the sharpened tip based on factors such as, for example, the depth of insertion desired for the sensor, the mechanical properties of the sensor substrate 904, or the parameter to be sensed by the chip 905, if any.

In the illustrated embodiment, the protector 907 is configured to rest against the upper surface (also called a traction surface 920, shown in FIG. 10B) of the chip/membrane combination prior to insertion. In an aspect, the chip/membrane combination is, or includes, a bump attached to the flexible sensor. When the sharp module 910 is fully inserted into the sensor module and before the needle is retracted, the distal end of the protector 907 rests near or against the subject's skin, the chip is inserted into the subject's body such that the upper surface is approximately flush with the subject's skin, and the sensor tip is inserted into the subject's body to a depth approximately equal to the distance between the upper surface and the sensor tip. The U-shaped protector 907 pushes on the traction surface 920 during the insertion process, applying an insertion force to the sensor 902 near the sharpened sensor tip.

Due to the skin's elasticity and friction between the needle and surrounding tissue, the skin-needle contact point is pushed inward by the tip of the needle 902 even after it punctures the skin. The inward force creates a gap between the lower surface of the sensor module 930 and the traction surface 920, providing a temporary space for the metal sheath 907. When the needle 902 and sheath 907 are retracted, the force pushing the skin inward is removed and the skin rebounds to close the gap while the sensor continues to insert deeper into the skin. Insertion of the sensor tip is completed only after the needle is fully retracted and the skin has retracted to its rest position.

In alternative embodiments, as shown in FIG. 11A, the chip 905 and protective membrane 903 are positioned on the flexible elongate sensor 1004 spaced a distance ‘d’ from the end of the U-shaped protector. In these embodiments, the distance ‘d’ may be selected such that the U-shaped protector does not contact or push the chip/membrane 905/903 during insertion. In these embodiments, the presence of the chip/membrane 905/903 may still aid insertion by stiffening the distal portion of the flexible elongate sensor 1004, increasing its propensity to be drawn into the opening created by the needle 902.

In other alternative embodiments, as shown in FIG. 11B, the chip 905 may be omitted, and replaced by a purely mechanical element 1020, also called a stiffener, to form the bump for pushing by the U-shaped protector. A purely mechanical feature 1020 can provide a traction surface 920 normal to the direction of insertion, equivalent to that provided by the combination of the chip 904 and protective membrane 903 shown in FIG. 10B. In an alternative, or in addition, the mechanical element 1020 may function as a stiffener to stiffen a distal end of the flexible elongate sensor 1004. The mechanical element 1020 may be formed from any suitable material, for example a biocompatible polymer that will bond well to the sensor 902 while not interfering with its sensing function or as part of its sensing function. Whether provided by a purely mechanical object 1020 attached near the sensor tip, or by a chip sensor 905, the combination of the traction surface 920 or stiffener element 1020 and the cooperating U-shaped protector 907 may improve the insertion success rate for the sensor insertion component 900. Likewise, stiffening the distal portion of the sensor 904, 1004 by a chipped or chipless stiffener also contributes to improving the insertion success rate.

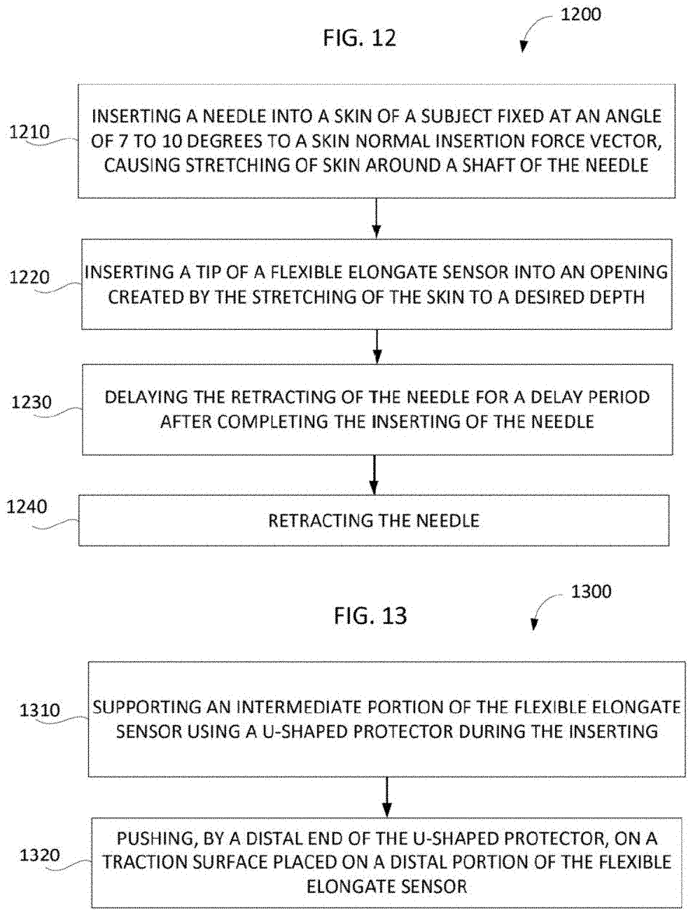

In summary of the foregoing, and by way of additional example, a method 1200 for inserting a distal portion of an analyte sensor into a subject using a sensor insertion component of an applicator as described herein, or equivalent apparatus, is shown in FIG. 12. The method 1200 may include, at 1210, inserting a needle into a skin of a subject fixed at an angle of 7 to 10 degrees, alternatively between about 6 to about 11 degrees, alternatively between about 5 to about 12 degrees, alternatively between about 4 to about 13 degree, alternatively greater than about 7 degrees, alternatively less than about 10 degrees, to a skin normal insertion force vector, causing stretching of skin around a shaft of the needle. The method 1200 may further include, at 1220, inserting a tip of a flexible elongate sensor into an opening created by the stretching of the skin to a desired depth. In another aspect, at 1230, the retracting of the needle is performed after a delay period after the inserting of the needle is completed. The delay period may be any suitable value, for example, between 0.3 and 3 seconds, or 1 second. The needle is kept fully inserted while the flexible elongate sensor moves into its inserted position. Then as the needle is retracted, the surrounding tissue rebounds while the sensor remains behind, causing the depth of sensor insertion relative to the skin surface to increase. The method 1200 may further include, at 1240, retracting the needle from the subject's skin, leaving behind the distal end of the elongate sensor at the desired depth below the surface of the skin.

FIG. 13 shows additional aspects 1300 that may optionally be included in the method 1200. In one optional aspect 1310, inserting the tip of the flexible elongate sensor may further include supporting an intermediate portion of the flexible elongate sensor using a U-shaped protector during the inserting. In another optional aspect 1320, inserting the tip of the flexible elongate sensor may further include pushing, by a distal end of the U-shaped protector, on a traction surface placed on a distal portion of the flexible elongate sensor.

Further details and aspects of the method 1200 should be apparent from the description of the various sensor insertion components and their modes of operation described herein above.

In some embodiments, methods of manufacturing sharp modules are described that include pre-orienting the needles and protectors in the sharp module. Exemplary methods of high-throughput manufacturing are described in U.S. Patent Publ. No. 2021/0308009, which is hereby expressly incorporated by reference in its entirety for all purposes.

In some embodiments, the needle assemblies described herein may contain a plurality of needles connected to a continuous support material via at least a plurality of first injection molded couplers. As used herein, the term “continuous support material” refers to a material whose length is much longer than its width, such as a material available in rolled form and having an aspect ratio of at least about 10, at least about 100, at least about 1,000, or at least about 10,000. Manufacturing processes employing a continuous support material may convey the continuous support material from a first reel to a second reel, with needles becoming connected (coupled) to the continuous support material in between the first and second reels. A continuous support material may facilitate fabrication of the needle assemblies disclosed herein via high-throughput manufacturing methods. It is to be appreciated, however, that the needle assemblies and processes of the present disclosure may be alternately formed or conducted with support materials having finite dimensions, such that the needle assemblies are manufactured in shorter lengths (discrete units) as well.

More specifically, the needle assemblies and processes described herein feature needles and protectors that are individually oriented within a plurality of apertures defined in a support material prior to an injection molding operation that connects the needles to the support material. In some embodiments, orientation of the needles and protectors within the needle assemblies may take place offline (prior to a manufacturing process incorporating a needle in a sensor inserter) to provide a stockpile of oriented needles. For example, robotic or manual ‘pick and place’ techniques may be used to provide an initial orientation of the needles and protectors prior to forming the needle assemblies as described herein. Once the needles and protectors have been connected to the support material with consistent orientation and spacing, further processing of the needle assemblies in a subsequent or contiguous production line may be readily conducted. As such, the present disclosure may facilitate high-throughput production of analyte sensors that are capable of insertion into a tissue of interest with minimal trauma, thereby allowing various user benefits to be realized.

In various embodiments, needle assemblies of the present disclosure may comprise: a support material having a plurality of apertures defined therein, and a first injection molded coupler located within each aperture that surrounds a proximal portion of a protector and connects the protector to a first location upon the support material. A needle may be coupled with the protector and held in a pre-determined orientation with respect to a longitudinal axis of the protector and/or the first injection molded coupler. As used herein, the term “distal portion” refers to a location upon the shaft of a needle that is nearer to the sharpened tip (i.e., the insertion tip), and the term “proximal portion” refers to a location upon the shaft of a needle that is nearer to the end opposite the insertion tip. As used herein, the term “distal portion” includes a segment of the needle that includes at least the insertion tip, and the term “proximal portion” includes a segment of the needle that includes the end opposite the insertion tip.

In some embodiments, each needle within the needle assemblies may be held in substantially the same orientation, within manufacturing tolerances. In some or other more specific embodiments, the needles in adjacent apertures may be spaced apart from one another substantially uniformly, within manufacturing tolerances. Angular deviation (variance) between the plurality of needles in the needle assemblies may be about 1 degree or less, or about 0.5 degrees of less, or about 0.25 degrees or less. According to various embodiments, the pitch (spacing between adjacent needles) may be about 15 mm or less, or about 12 mm or less, or about 10 mm or less, or about 7 mm or less, or about 5 mm or less, with a pitch variance of about 0.02 mm or less. In more specific embodiments, the pitch may constitute a spacing between about 8 mm and about 10 mm, with a pitch variance of about 0.02 mm or less. In some or other embodiments, the length of the needles may be about 20 mm or less, or about 15 mm or less, or about 12 mm or less, or about 10 mm or less, or about 8 mm or less, with a length variance of about 0.05 mm or less. In more specific embodiments, the length of the needles may range between about 9 mm and about 12 mm, or between about 10 mm and about 11 mm, with a length variance of about 0.05 mm or less.

According to some embodiments, the needle within each aperture may be held non-parallel with respect to the longitudinal axis of the protector. In more specific embodiments, the needle within each aperture may be held at an angle ranging between about 5° and about 15°, or between about 7° and about 12°, or between about 8° and about 11°, with respect to the longitudinal axis, including any value or sub-range therebetween. By angling the needle, the skin may stretch to one side when making a skin penetration, which creates a gap for promoting easier sensor insertion. In still more specific embodiments, the needle within each aperture may be held at an angle ranging between about 9° and about 10° with respect to the longitudinal axis, including any value or sub-range therebetween.

In certain embodiments, the needle assemblies described herein may further comprise a second injection molded coupler located within each aperture that surrounds a distal portion of the needle and connects the needle to a second location upon the support material. The second injection molded coupler may aid in protecting the insertion tip of the needle during fabrication of the needle assemblies described herein, thereby potentially lowering the fraction of units rejected for quality control defects during subsequent analyte sensor inserter fabrication. Moreover, the second injection molded coupler may further stabilize the needle within each aperture by limiting flexural motion during fabrication of the needle assemblies. Alternately, a second injection molded piece may surround a distal portion of the needle but remain unattached (no coupling) to the support material. Such configurations may similarly aid in protecting the insertion tip of the needle.

Various methods for fabricating and using the needle assemblies of the present disclosure are also contemplated herein. Methods for using the needle assemblies may include separating individual needles arranged in a defined orientation within a needle construct and incorporating the oriented needles into an analyte sensor inserter, as described in further detail below.

In some embodiments, methods for fabricating the needle assemblies of the present disclosure may comprise: providing a support material having a plurality of apertures defined therein, a neck extending from the support material into each aperture; coupling an elongate protector having a channel to the neck extending from the support material into each aperture; coupling a needle to the elongate protector in each aperture; and injection molding polymeric material to form a first injection molded coupler that surrounds both the neck and a proximal portion of the elongate protector within each aperture, thereby connecting the needle to a first location upon the support material via the neck. The needle assemblies may be fabricated such that the neck is coincident with a longitudinal axis of the first injection molded coupler, and such that the needle is held in a pre-determined orientation with respect to the longitudinal axis.

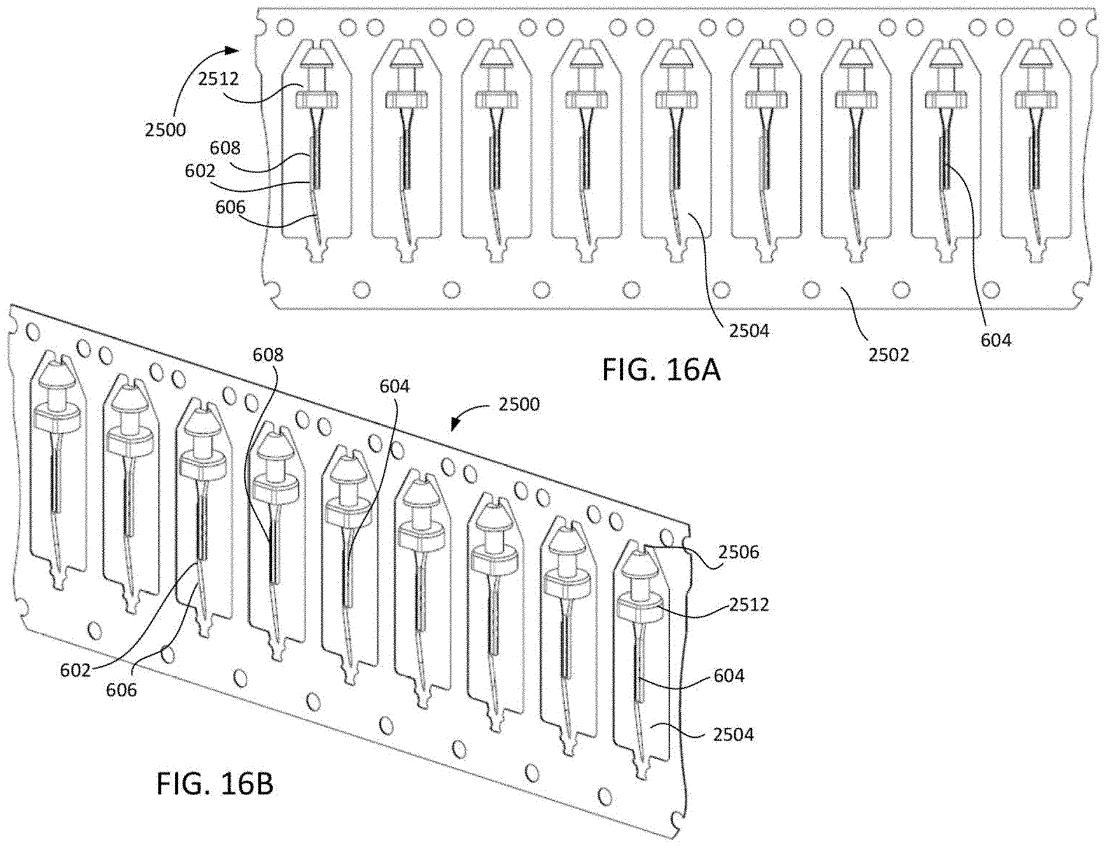

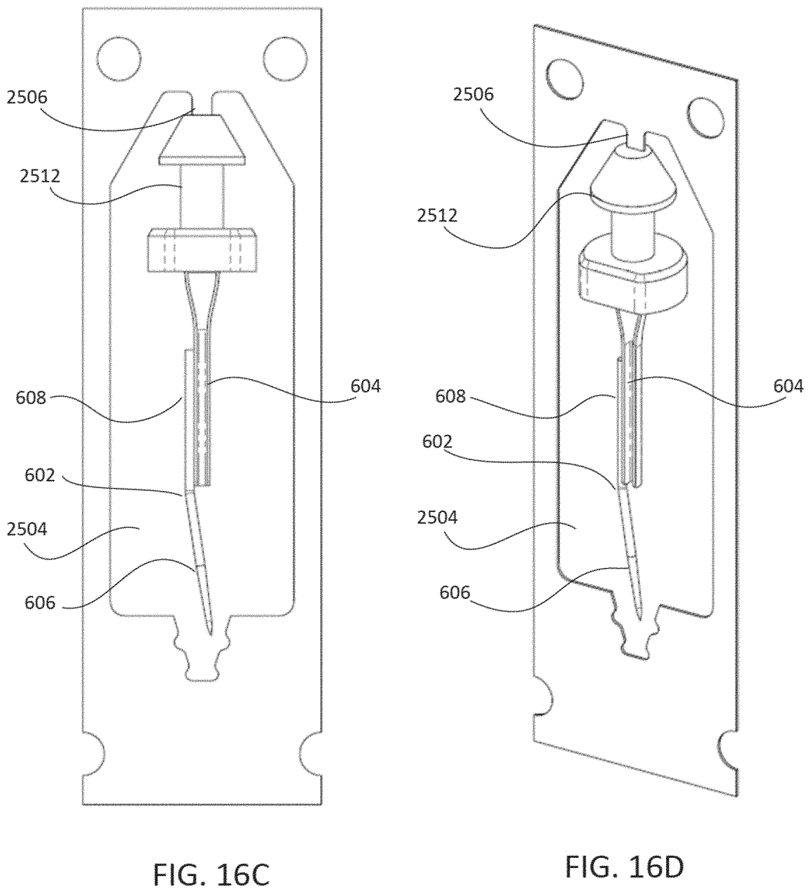

In some embodiments, methods for fabricating the needle assemblies of the present disclosure may comprise: providing a support material comprising a frame comprising a plurality of apertures defined therein, an elongate protector in each of the plurality of apertures, and a neck extending from the frame to the elongate protector in each of the plurality of apertures; attaching a sharp to each of the elongate protectors, wherein the sharp comprises a proximal portion, a distal portion, and a bent portion between the proximal and distal portion, wherein the proximal portion of the sharp is attached to the elongate protector and the distal portion of the sharp is not attached to the elongate protector; and injection molding polymeric material to form a first injection molded coupler that surrounds a portion of the neck and a proximal portion of the elongate coupler, and wherein the distal portion of the sharp extends past a distal end of the elongate protector at an angle to the longitudinal axis of the elongate protector. The needle assemblies may be fabricated such that the neck is coincident with a longitudinal axis of the first injection molded coupler, and such that the needle is held in a pre-determined orientation with respect to the longitudinal axis, such as shown above in FIGS. 16A-16D.

In some embodiments, a section of the continuous support material, including the frame, necks, and elongate protectors, may be placed within a mold for injection molding.

In more specific embodiments, the support material may comprise a continuous support material, such as a continuous metal tape.

According to some further embodiments, methods for fabricating needle assemblies of the present disclosure may further comprise injection molding polymeric material to form a second injection molded coupler that surrounds a distal portion of the needle within each aperture and connects the needle to a second location upon the support material. Alternately, a second injection molded coupler (injection molded piece) surrounding a distal portion of the needle within each aperture may be fabricated similarly, but without making a connection to the support material.

Injection molding processes suitable for forming the first and second injection molded couplers will be familiar to one having ordinary skill in the art. Such processes may comprise placing one or more molds within each aperture, and injecting polymeric material into the mold(s) to form the first injection molded coupler and optionally the second injection molded coupler, wherein each injection molded coupler is positioned as described above. The first and second injection molded couplers may be formed in the same injection molding process or in separate injection molding processes. Moreover, the polymeric material used for forming the first injection molded coupler and the second injection molded coupler may be the same or different. Any suitable thermoplastic or thermosetting polymeric material may be used to form the first and second injection molded couplers. For example, in some embodiments, the first injection molded coupler may be formed from a rigid polymeric material that may facilitate use of a needle construct in an analyte sensor inserter, and the second injection molded coupler may be formed from a compliant polymeric material that may facilitate needle withdrawal at a desired time. The injection molding processes may further comprise placing an elongate protector, e.g., an elongate U-shaped protector, that is coupled with a needle within each mold prior to injecting polymeric material thereto. In some embodiments, manual or automated pick and place techniques may be used for positioning the elongate protector within the mold(s).

Methods for fabricating the needle assemblies of the present disclosure may further comprise, in some embodiments, die-cutting or stamping the support material to define the plurality of apertures. The apertures may be of a desired size and shape to contain the needle, the elongate protector, and at least the first injection molded coupler. Suitable die-cutting or stamping processes will be familiar to one having ordinary skill in the art. The die-cutting or stamping process may be conducted integrally with the injection molding process(es) or in a separate production line before the injection molding process(es). In other embodiments, the support material may be obtained, sourced, or purchased with a plurality of apertures already being defined therein.

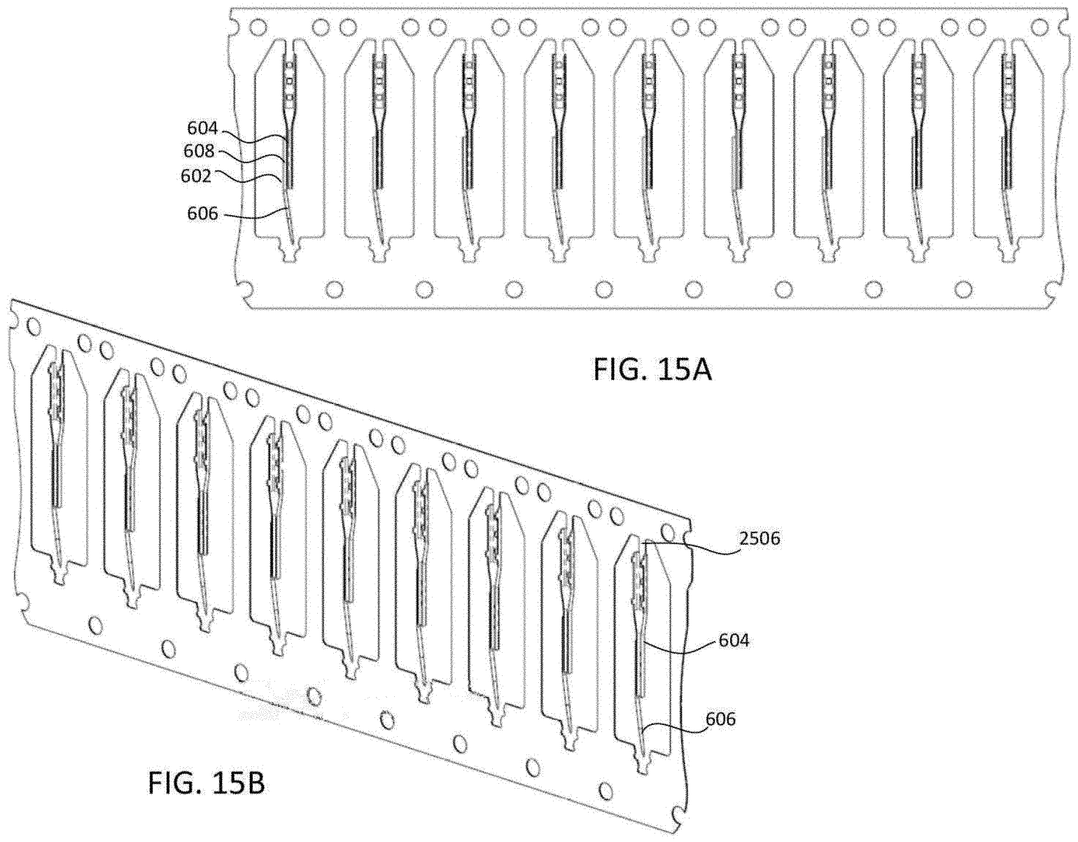

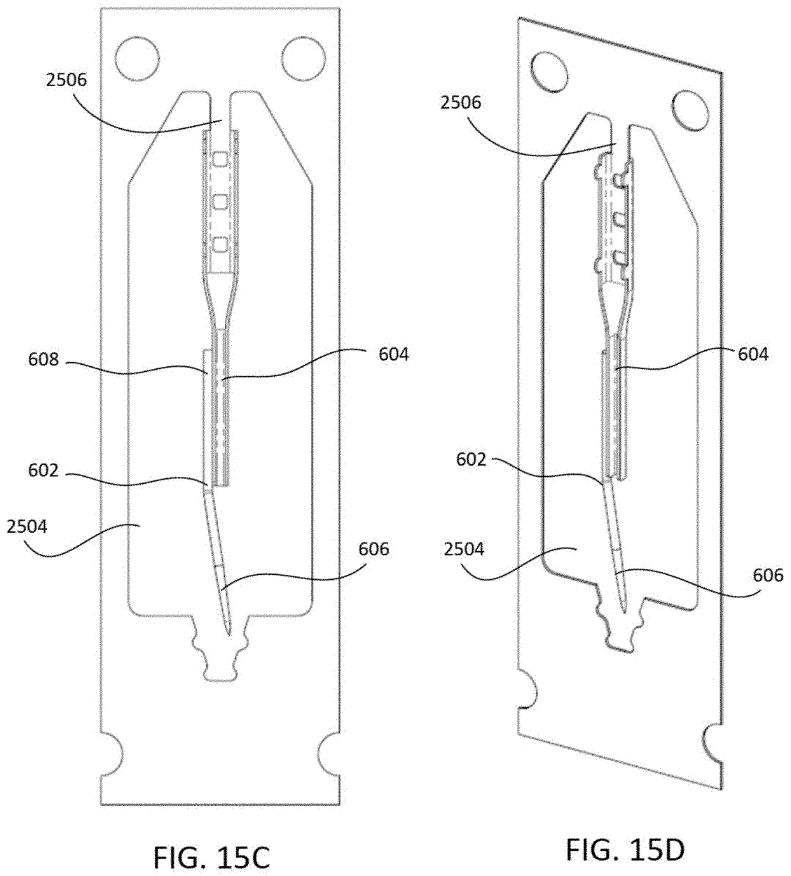

FIGS. 14-17 show diagrams illustrating exemplary stages of a process whereby a first configuration of needle assemblies of the present disclosure may be fabricated. In the interest of clarity, FIGS. 14A-14B, 15A15-B, 16A-16B, and 17A-17B show needle assembly fabrication taking place in nine apertures, but it is to be appreciated that the depicted concepts may be extended to fabrication taking place in more than nine apertures of a support material, either simultaneously or non-simultaneously (consecutively).

In FIGS. 14A-14B, continuous support material 2502, e.g., a continuous metal tape having a plurality of apertures 2504 of defined shape is obtained/provided (e.g., as a pre-punched tape from a commercial source) or formed (e.g., by stamping or die-cutting) prior to fabricating a needle assembly.

FIGS. 14A-14D show a diagram of a portion of a needle assembly of the present disclosure having a plurality of elongate protectors 604 oriented in a vertical orientation in each aperture 2504 of the continuous support material 2502. Continuous support material 2502 may be a continuous metal tape, strip or film, although alternative materials and forms capable of being processed in a reel-to-reel manner may also be used in some cases. In some embodiments, stainless steel may be a suitable metal upon which needle assembly may be fabricated. Other suitable tapes may include alternative metals or other materials that are able to withstand the injection molding temperatures needed to fabricate needle assembly. In alternative embodiments, needle assembly may be formed upon a support material having finite length, such that assembly fabrication takes place in discrete units rather than by reel-to-reel continuous processing.

A neck 2506 extends as an elongate member into each aperture 2504 from a frame of the continuous support material 2502. A longitudinal axis of the elongate protector 604 may be parallel and/or in line with a longitudinal axis of the neck 2506. A longitudinal axis of the elongate protector 604 may be about parallel to a skin normal insertion force vector. The continuous support material 2502, neck 2506, and elongate protector 604 may be made from a single piece of material. In some embodiments, a metal etching or die cutting process may be used to form frames with flat metal pieces needed to form the elongate protectors 604 in each of the plurality of apertures 2504 in the continuous support material 2502. The elongate protectors 604 may be formed using a stamping process, as is well known in the art. Alternatively, in other embodiments, the elongate protector may be a separate piece and a proximal portion of the elongate protector 604 may be coupled with a distal portion of the neck 2506.

Next, as seen in FIGS. 15A-15D, a pre-bent needle 602 may be connected to a distal portion of the elongate protector 604. In some embodiments, the pre-bent needles 602 may be coupled or attached to the distal portion of the elongate protector 604 by laser spot welding. As seen in FIGS. 15C-15D, the elongate protector 604 may have a first side having a U-shaped channel and a second side, wherein the second side is the back side of the U-shaped channel. The pre-bent needle 602 may have a proximal portion 608, a bent portion, and a distal portion 606. The bent portion may be located between the proximal and distal portions 608, 606, and may comprise a single bend, angle, or deflection in the bent portion. The proximal and distal portions may connect and form a single angle in the bent portion. The single angle may be an obtuse angle. The single angle may be between about 160° and about 175°, alternatively between about 165° and about 175°. In some embodiments, a proximal portion 608 of the needle may be attached to a distal portion of the elongate protector on the second side.

Next, a mold (not shown) may be arranged within each aperture 2504 in preparation for injection molding. Neck 2506 extends into the mold so that a connection between continuous support material 2502, elongate protector 604, and needle 602 occurs upon injection molding to form first injection molded coupler 2512 (or base component). As seen in FIGS. 16A-16D, first injection molded coupler 2512 surrounds a portion of neck 2506 and a portion of the elongate protector 604. A second connection between continuous support material 2502 and needle 602 may also be present in a distal portion 606 of the needle as well.

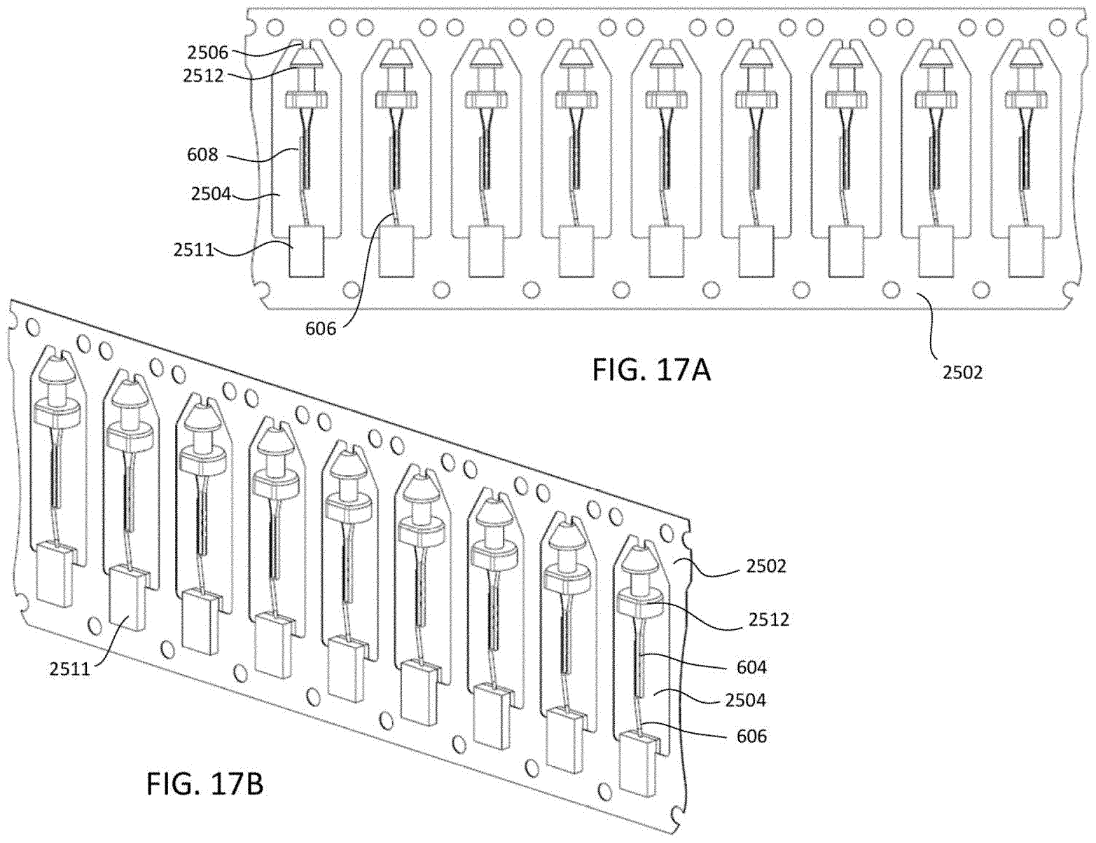

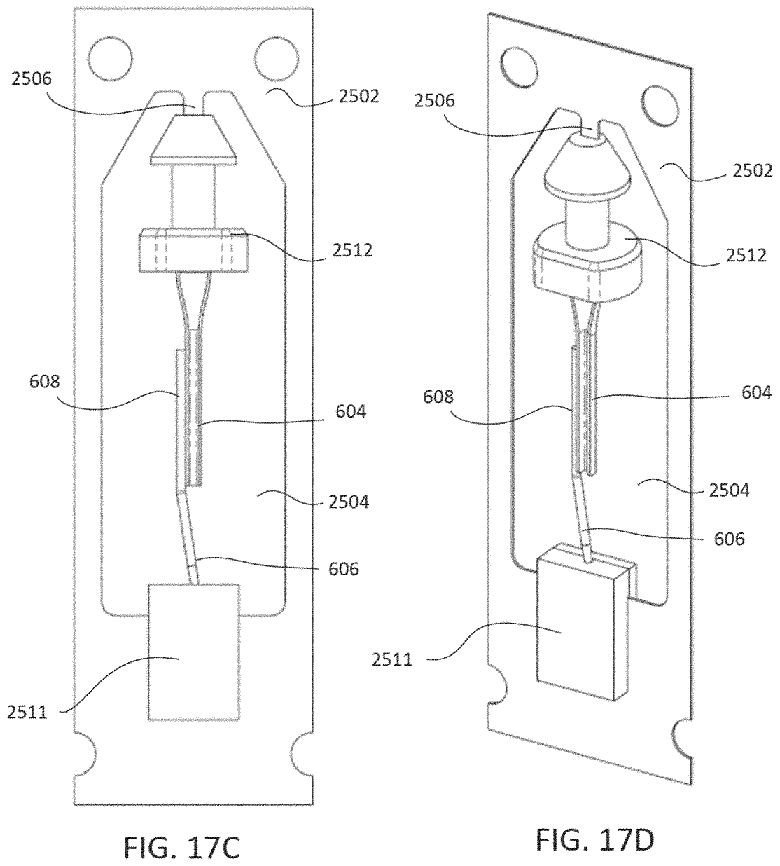

FIGS. 16A-16D illustrate exemplary needle assemblies 2500 in which the injection molding operation(s) have been completed and the mold(s) has been removed from each aperture 2504. Following the injection molding operation(s), needle 602 is connected to continuous support material 2502 at neck 2506 via elongate protector 604 and first injection molded coupler 2512, and optionally via second injection molded coupler 2514 (see FIGS. 17A-17D) at the bottom of aperture 2504. A second injection molded coupler may be removably connected to needle 602, as described above, to aid in protecting the insertion tip during assembly fabrication and needle manipulation.

In some embodiments, a single mold that encompasses the distal end of the neck that is coupled with the proximal end of the elongate protector and the needle may be utilized instead of two separate molds for the proximal and distal ends of the needle assembly. Alternately, the distal portion 606 of needle 602 may reside outside of the mold and remain unsupported throughout the injection molding process.

Each mold may have a shape complementarity with each aperture 2504, such that each mold fits therein and overlays one or more desired portions of continuous support material 2502. As seen in FIGS. 17A-17D, when a second mold 2511 is used for forming the second injection molded coupler, it may similarly overlay a bottom portion of continuous support material 2502. According to some embodiments, the mold may be a two-piece mold to facilitate the loading of the needle therein. In such embodiments, a first piece of the mold may be positioned adjacent to a first side of continuous support material 2502, and a second piece of the mold may be positioned adjacent to a second side of continuous support material 2502. One or more cavities may be defined between the two pieces. The one or more cavities may be bisected by the plane of continuous support material 2502. Neck 2506 may extend into at least one of the cavities such that injection molding forms a connection between the elongate protector 604, needle 602, and neck 2506. The two pieces (hemispheres) of the mold may be assembled together in aperture 2504 in preparation for injection molding. Although the mold may be a two-piece mold to facilitate loading of the needle, it is to be appreciated that the mold may also be a one-piece mold in some alternative embodiments.

The mold contains cavities therein may be filled with a thermoplastic or thermosetting material during a single injection molding process or separately during two or more injection molding processes to define a first injection molded coupler 2512 (see FIGS. 16A-16D) and a second injection molded coupler covering a distal tip of the needle see FIGS. 17A-17D). Neck 2506 extends into the cavity of the mold, such that first injection molded coupler 2512 (see FIGS. 16A-16D) is formed in the cavity and surrounds neck 2506 and a portion of first injection molded coupler 2512. Similarly, a second injection molded coupler may be formed in a cavity of a separate or the same mold that contains the distal tip of the needle. The cavity forming the second injection molded coupler may overlay a corresponding notch in continuous support material 2502 at the bottom of aperture 2504, such that a first portion of the second injection molded coupler overlays the notch and a second portion is formed upon continuous support material 2502. Alternatively, the second injection molded coupler (injection molded piece) may surround a distal portion of needle but not form a connection to continuous support material 2502.

For a single mold configured to form the first and second injection molded couplers, a channel may extend along a length of the mold between a proximal cavity configured to form the first injection molded coupler and a distal cavity configured to form the second injection molded coupler. The channel may be sized to receive the elongate protector 604 and needle 602 such that distal portion 606 of needle extends into the distal cavity and a proximal portion of the elongate protector extends into the proximal cavity. Once injection molding has taken place to form the first injection molded coupler and second injection molded coupler, needle 602 is connected to continuous support material 2502 both distally and proximally and held in a pre-determined orientation for further manipulation. The channel is generally not filled with thermoplastic or thermosetting material during the injection molding operation(s).

As discussed above, the distal portion of the needle may also be unsupported, as shown for the needle assemblies in FIGS. 16A-16D. A mold omitting the channel for the needle and elongate protector and the distal cavity may be used when forming a needle assembly with the needle having an unsupported distal portion.

Once injection molding is complete and each mold has been removed, each needle assembly 2500 may be stored for further use or fed directly into a process for fabricating an analyte sensor inserter (see, e.g., sensor applicator 150 of FIG. 1). In either case, the position of each needle 602 remains fixed with respect to first injection molded coupler 2512 until further needle manipulation takes place, as described hereinbelow. In addition, the separation and orientation of each needle remains fixed with respect to one another, also facilitating further needle manipulations. In more specific embodiments, each needle may be spaced apart substantially uniformly. Because the needle assembly 2500 provides a highly ordered and regular arrangement of multiple needles, they may be manipulated in a manner similar to that of conventional arrays of larger gauge needles or similar sharps. As such, the needle assemblies of the present disclosure may facilitate various manufacturing processes using only minor modifications of existing production lines, as described hereinafter. Namely, the needle assemblies of the present disclosure may directly replace an array of larger gauge needles or similar sharps used in present manufacturing processes.

Prior to incorporation in an analyte sensor inserter or other type of device, individual needles are removed from needle assembly 2500 in the form of a needle construct. The needle construct comprises needle 602, elongate protector 604, and first injection molded coupler 512, such that the needle 602 remains held in a pre-determined orientation with respect to the longitudinal axis of first injection molded coupler 2512, particularly non-parallel orientations with respect to the longitudinal axis. Removal of individual needle constructs may take place as a further operation of forming needle assembly or as an entirely separate process, according to various embodiments.

Accordingly, in further embodiments, methods of the present disclosure may comprise separating a needle construct from the support material, such as a continuous metal tape, and the second injection molded coupler, if present, and incorporating the needle construct into an insertion device for an analyte sensor or another type of device. The needle construct includes a needle, elongate protector, and the first injection molded coupler, wherein the first injection molded coupler surrounds a proximal portion of the elongate protector. The needle construct may optionally include a distal portion of the elongate protector coupled with a proximal portion of the needle.

In further embodiments, separating the needle construct may comprise severing the neck 2506 adjacent to the first injection molded coupler 2512, and pulling the distal end 606 of the needle from the second injection molded coupler, where present. In embodiments where the second injection molded coupler is not present, severing the neck adjacent to the first injection molded coupler directly releases the needle construct from the needle assembly. As described in U.S. Patent Publ. No. 2021/0308009, which was previously incorporated by reference in its entirety for all purposes, severing the neck to release the needle construct leaves a metal core within the first injection molded coupler, where the metal core may be coincident with the longitudinal axis of the first injection molded coupler. Once separated from the needle assembly, the individual needle constructs may be further manipulated into a production line.

The neck 2506 may be severed to break the first connection to continuous support material 2502. Severing of neck 2506 may take place using any suitable method, such as guillotine cutting, die cutting, scissor cutting, or the like. Application of a gentle axial pulling force along a longitudinal axis may be sufficient to dislodge the needle from the second injection molded coupler if present, thereby freeing the needle construct. Similar operations may be used to separate needle construct in embodiments either lacking a second injection molded coupler or having a second injection molded coupler that is unconnected to the support material 2502.

A number of deflectable structures are described herein, including but not limited to a hybrid needle assembly, with or without a chip sensor or mechanical traction surface. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened).

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. Features, elements, components, functions, and steps may be combined from different embodiments to provide new combinations. Likewise, features, elements, components, functions, and steps from one embodiment may be substituted and combined with those of another, even where the foregoing description does not explicitly disclose that such combinations or substitutions are possible. Such combinations and substitutions should be apparent to those of ordinary skill in the art without exhaustive disclosure of every conceivable possibility.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Aspects of the invention are set out in the independent claims and preferred features are set out in the dependent claims. Preferred features of each aspect may be provided in combination with each other within particular embodiments and may also be provided in combination with other aspects.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, a sensor insertion component for use in an applicator of an in vivo analyte sensor includes a sensor module holding a connector coupled with a proximal end of a flexible elongate sensor, wherein the sensor module comprises at least one surface defining a skin normal insertion force vector; and a sharp module held by the sensor module and configured for motion relative to the sensor module parallel to the skin normal insertion force vector, wherein the sharp module comprises: a base configured for the motion relative to the sensor module; a U-shaped protector fixed to the base, having an intermediate portion of the flexible elongate sensor disposed along a length thereof with a distal portion of the flexible elongate sensor extending past a distal end thereof; and a sharp fixed to at least one of the base or the U-shaped protector, the sharp having an outer diameter not greater than 0.56 mm and a distal portion extending past a distal end of the flexible elongate sensor at an angle to the skin normal insertion force vector of not less than five degrees and not greater than fifteen degrees.

In some embodiments, wherein the sharp comprises a solid needle having a diameter not greater than about 0.5 mm. In some embodiments, the diameter of the sharp is less than or equal to about 0.35 mm.

In some embodiments, the motion is a sliding motion.

In some embodiments, the sharp is aligned within about 7° of the skin normal insertion force vector.

In some embodiments, the intermediate portion of the flexible elongate sensor is disposed in a channel of the U-shaped protector.

In some embodiments, the distal portion of the sharp has a length in a range of about 1.0 to about 5.0 mm.

In some embodiments, the distal portion of the flexible elongate sensor has a length in a range of about 0.5 to about 4.0 mm.

In some embodiments, the U-shaped protector has a length extending from the base in the range of about 1.0 to about 10 mm.

In some embodiments, the sharp is fixed to the base.

In some embodiments, the sharp is fixed to the U-shaped protector.

In some embodiments, a distal end of the flexible elongate sensor is sharpened to a point.

In some embodiments, a distal end of the flexible elongate sensor touches a shaft of the sharp.

In some embodiments, a distal end of the flexible elongate sensor is disposed along a shaft of the sharp.

In some embodiments, the insertion component also includes a bump having a traction surface attached to the flexible elongate sensor, with the traction surface disposed for engagement with the distal end of the U-shaped protector for transmission of an insertion force along the skin normal insertion force vector to the flexible elongate sensor. In some embodiments, the bump comprises a sensor chip. In some embodiments, the sensor chip is encased in a protective membrane. In some embodiments, the sensor chip is coupled with the connector by a conductor disposed along the flexible elongate sensor. In some embodiments, the sensor chip comprises a thermistor.

In some embodiments, the insertion component also includes a stiffener coupled with a distal portion of the flexible elongate sensor. In some embodiments, the stiffener provides a sensing function.

In many embodiments, a method for inserting a distal portion of an analyte sensor into a subject using an applicator includes the steps of: inserting a needle into a skin of a subject fixed at an angle of about 5 to about 15 degrees to a skin normal insertion force vector, causing stretching of skin around a shaft of the needle; inserting a tip of a flexible elongate sensor into an opening created by the stretching of the skin to a desired depth and waiting for a delay period; and retracting the needle after the delay period.

In some embodiments, inserting the tip of the flexible elongate sensor further comprises supporting an intermediate portion of the flexible elongate sensor using a U-shaped protector during the inserting.

In some embodiments, the method further includes the steps of pushing, by a distal end of the U-shaped protector, on a traction surface placed on a distal portion of the flexible elongate sensor.

In some embodiments, the method is performed using the sensor insertion component of claim 1.

In some embodiments, the delay period is between 0.5 and 3 seconds.

In some embodiments, the delay period is 1 second.

In many embodiments, a sensor insertion component for use in an applicator of an in vivo analyte sensor includes: a sensor module comprising a connector coupled with a sensor; and a sharp module coupled with the sensor module, the sharp module comprising: a base; an elongate protector coupled with the base, the elongate protector comprising a longitudinal axis and a channel configured to receive an intermediate portion of the sensor disposed along a length thereof with a distal portion of the sensor extending past a distal end thereof; and a sharp coupled with the elongate protector or the base, the sharp comprising a proximal portion and a distal portion, wherein the distal portion extends past a distal end of the elongate protector at an angle to the longitudinal axis of the elongate protector, wherein the angle is between about 5° and about 15°.

In some embodiments, the distal portion of the sharp is not attached to the elongate protector.

In some embodiments, the proximal portion of the sharp is attached to the elongate protector.

In some embodiments, the sharp further comprises a bent portion between the proximal and distal portion. In some embodiments, the bent portion comprises a single deflection having an angle formed by the proximal and distal portions of the sharp, wherein the angle is between about 160° and about 175°.

In some embodiments, the elongate protector has a first side and a second side, wherein the first side comprises the channel, and the proximal portion of the sharp is attached to the second side of the elongate protector.

In some embodiments, the channel of the elongate protector is U-shaped.

In some embodiments, the channel extends along a distal portion of the elongate protector.

In some embodiments, the channel does not extend along a proximal portion of the elongate protector.

In some embodiments, the sharp comprises a solid needle having a diameter not greater than about 0.5 mm.

In some embodiments, the angle is about 7°.

In some embodiments, the distal portion of the sharp has a length in a range of about 1.0 to about 5.0 mm.

In some embodiments, the sensor further comprises a bump having a traction surface disposed for engagement with a distal end of the elongate protector for transmission of an insertion force along an insertion force vector that is substantially parallel to the longitudinal axis of the elongate protector. In some embodiments, the bump comprises a sensor chip. In some embodiments, the sensor chip is encased in a protective membrane.

In some embodiments, the sensor further comprises a stiffener coupled with a distal portion of the sensor.

In many embodiments, a method for inserting a distal portion of an analyte sensor into a subject using an applicator includes the steps of: positioning an applicator against a skin surface of the subject, the applicator comprising a housing, a sensor module comprising a connector coupled with a proximal end of a sensor, and a sharp module coupled with the sensor module, wherein the sharp module comprises: a base; an elongate protector coupled with the base, the elongate protector comprising a longitudinal axis and a channel configured to receive an intermediate portion of the sensor disposed along a length thereof with a distal portion of the sensor extending past a distal end thereof; and a sharp coupled with the elongate protector or the base, the sharp comprising a proximal portion and a distal portion, wherein the distal portion extends past a distal end of the elongate protector at an angle to the longitudinal axis of the elongate protector, wherein the angle is between about 5° and about 15°; inserting a distal end of the sharp into a skin of the subject by applying a force against a proximal portion of the housing, wherein the angle of the distal portion causes stretching of skin around a shaft of the needle and creates an opening; inserting a distal end of the sensor into the opening; and retracting the sharp.

In some embodiments, the distal portion of the sharp is not attached to the elongate protector.

In some embodiments, the proximal portion of the sharp is attached to the elongate protector.

In some embodiments, the sharp further comprises a bent portion between the proximal and distal portion.

In some embodiments, the bent portion comprises a single deflection having an angle formed by the proximal and distal portions of the sharp, wherein the angle is between about 160° and about 175°.

In some embodiments, the elongate protector has a first side and a second side, wherein the first side comprises the channel, and the proximal portion of the sharp is attached to the second side of the elongate protector.

In some embodiments, the channel of the elongate protector is U-shaped.

In some embodiments, the channel extends along a distal portion of the elongate protector.

In some embodiments, the channel does not extend along a proximal portion of the elongate protector.

In some embodiments, the sharp comprises a solid needle having a diameter not greater than about 0.5 mm.

In some embodiments, the angle is about 7°.

In some embodiments, the distal portion of the sharp has a length in a range of about 1.0 to about 5.0 mm.

In many embodiments, a needle assembly includes: a support material having a plurality of apertures defined therein; an elongate protector located in each aperture of the plurality of apertures, the elongate protector comprising a U-shaped channel, a longitudinal axis, a proximal end, and a distal end, wherein the distal end is coupled with the support material; a first injection molded coupler located within each aperture of the plurality of apertures, wherein the first injection molded coupler surrounds the proximal end of the elongate protector and a portion of the support material; and a sharp located in each aperture of the plurality of apertures, the sharp comprising a proximal portion, a distal portion, and a bent portion between the proximal and distal portion, wherein the proximal portion of the sharp is coupled with the elongate protector and the distal portion of the sharp is not attached to the elongate protector.

In some embodiments, the support material comprises a continuous metal tape.

In some embodiments, the bent portion comprises a single deflection.

In some embodiments, a neck extends from the support material into each of the plurality of apertures and connects to the elongate protector, and wherein the first injection molded coupler surrounds the neck and a proximal end of the elongate protector. In some embodiments, the neck has a longitudinal axis and wherein the longitudinal axis of the neck is parallel to the longitudinal axis of the elongate coupler.

In some embodiments, the assembly further includes a second injection molded coupler located within each aperture of the plurality of apertures. wherein the second injection molded coupler surrounds a distal end of the sharp. In some embodiments, the second injection molded coupler connects the sharp to a second portion of the support material.

In some embodiments, the first injection molded coupler does not surround a proximal end of the sharp.

In some embodiments, the distal portion of the sharp is held at an angle ranging between about 5° and about 15° to the longitudinal axis of the elongate coupler.

In some embodiments, the distal portion of the sharp is held non-parallel with respect to the longitudinal axis of the elongate coupler.

In many embodiments, a method includes the steps of: providing a support material comprising a frame comprising a plurality of apertures defined therein, an elongate protector in each of the plurality of apertures, and a neck extending from the frame to the elongate protector in each of the plurality of apertures; attaching a sharp to each of the elongate protectors, wherein the sharp comprises a proximal portion, a distal portion, and a bent portion between the proximal and distal portion, wherein the proximal portion of the sharp is attached to the elongate protector and the distal portion of the sharp is not attached to the elongate protector; and injection molding polymeric material to form a first injection molded coupler that surrounds a portion of the neck and a proximal portion of the elongate coupler, wherein a longitudinal axis of the neck is parallel to a longitudinal axis of the elongate coupler, and wherein the distal portion of the sharp extends past a distal end of the elongate protector at an angle to the longitudinal axis of the elongate protector, wherein the angle is between about 5° and about 15°.

In some embodiments, the bent portion of the sharp comprises a single deflection.

In some embodiments, the support material comprises a continuous metal tape.

In some embodiments, the method further includes the step of injection molding polymeric material to form a second injection molded coupler that surrounds a distal end of the sharp and connects the sharp to a second location on the support material.

In some embodiments, the method further includes the steps of separating a needle construct from the support material and the second injection molded coupler, the needle construct comprising the sharp, the elongate protector, and the first injection molded coupler; and incorporating the needle construct into an insertion device for an analyte sensor. In some embodiments, separating the needle construct comprises severing the neck adjacent to the first injection molded coupler.

In many embodiments, an applicator includes: a housing comprising a distal end configured to be placed against a skin surface; a sensor module comprising a connector coupled with a sensor; a sharp module coupled with the sensor module, the sharp module comprising: a base; an elongate protector coupled with the base; and a sharp coupled with the elongate protector or the base, wherein a distal portion of the sharp extends past a distal end of the elongate protector at an angle to a longitudinal axis of the elongate protector; a retraction spring configured to automatically retract the sharp module and the sharp from the skin surface in a proximal direction; and sensor electronics configured to be advanced from a proximal position in the housing to a distal position.

In some embodiments, the sharp comprises a proximal portion and a bent portion between the proximal portion and the distal portion.

In some embodiments, the elongate protector comprises a longitudinal axis and a channel configured to receive an intermediate portion of the sensor disposed along a length thereof with a distal portion of the sensor extending past a distal end thereof. In some embodiments, the channel is U-shaped.

In some embodiments, the distal portion of the sharp is not attached to the elongate protector. In some embodiments, the proximal portion of the sharp is attached to the elongate protector. In some embodiments, the bent portion comprises a single deflection having an angle formed by the proximal and distal portions of the sharp, wherein the angle is between about 160° and about 175°.

In some embodiments, the elongate protector has a first side and a second side, wherein the first side comprises the channel, and the proximal portion of the sharp is attached to the second side of the elongate protector.

In some embodiments, the channel of the elongate protector is U-shaped.

In some embodiments, the channel extends along a distal portion of the elongate protector.

In some embodiments, the distal portion of the sharp has a length in a range of about 1.0 to about 5.0 mm.

In some embodiments, the sharp comprises a solid needle having a diameter not greater than about 0.5 mm.

In some embodiments, the angle is between about 5° and about 15°.

In some embodiments, the applicator is configured to advance the sensor electronics in a distal direction.

In some embodiments, the sensor further comprises a bump having a traction surface disposed for engagement with a distal end of the elongate protector for transmission of an insertion force along an insertion force vector that is substantially parallel to the longitudinal axis of the elongate protector. In some embodiments, the bump comprises a sensor chip. In some embodiments, the sensor chip is encased in a protective membrane.

In some embodiments, the sensor further comprises a stiffener coupled with a distal portion of the sensor.

### Clauses

Exemplary embodiments are set out in the following numbered clauses.
Clause 1. An applicator, comprising:

a housing comprising a distal end configured to be placed against a skin surface;

a sensor module comprising a connector coupled with a sensor;

a sharp module coupled with the sensor module, the sharp module comprising:
a base;
an elongate protector coupled with the base; and
a sharp coupled with the elongate protector or the base, wherein a distal portion of the sharp extends past a distal end of the elongate protector at an angle to a longitudinal axis of the elongate protector;

a retraction spring configured to automatically retract the sharp module and the sharp from the skin surface in a proximal direction; and

sensor electronics configured to be advanced from a proximal position in the housing to a distal position.
Clause 2. The applicator of clause 1, wherein the sharp comprises a proximal portion and a bent portion between the proximal portion and the distal portion.
Clause 3. The applicator of clause 1 or 2, wherein the elongate protector comprises a longitudinal axis and a channel configured to receive an intermediate portion of the sensor disposed along a length thereof with a distal portion of the sensor extending past a distal end thereof.
Clause 4. The applicator of clause 3, wherein the channel is U-shaped.
Clause 5. The applicator of any of the preceding clauses, wherein the distal portion of the sharp is not attached to the elongate protector.
Clause 6. The applicator of clause 2, wherein the proximal portion of the sharp is attached to the elongate protector.
Clause 7. The applicator of clause 2, wherein the bent portion comprises a single deflection having an angle formed by the proximal and distal portions of the sharp, wherein the angle is between about 160° and about 175°.
Clause 8. The applicator of clause 3, wherein the elongate protector has a first side and a second side, wherein the first side comprises the channel, and the proximal portion of the sharp is attached to the second side of the elongate protector.
Clause 9. The applicator of clause 3, wherein the channel of the elongate protector is U-shaped.
Clause 10. The applicator of clause 3, wherein the channel extends along a distal portion of the elongate protector.
Clause 11. The applicator of any of the preceding clauses, wherein the distal portion of the sharp has a length in a range of about 1.0 to about 5.0 mm.
Clause 12. The applicator of any of the preceding clauses, wherein the sharp comprises a solid needle having a diameter not greater than about 0.5 mm.
Clause 13. The applicator of any of the preceding clauses, wherein the angle is between about 5° and about 15°.
Clause 14. The applicator of any of the preceding clauses, wherein the applicator is configured to advance the sensor electronics in a distal direction.
Clause 15. The applicator of any of the preceding clauses, wherein the sensor further comprises a bump having a traction surface disposed for engagement with a distal end of the elongate protector for transmission of an insertion force along an insertion force vector that is substantially parallel to the longitudinal axis of the elongate protector.
Clause 16. The applicator of clause 15, wherein the bump comprises a sensor chip.
Clause 17. The applicator of clause 16, wherein the sensor chip is encased in a protective membrane.
Clause 18. The applicator of any of the preceding clauses, wherein the sensor further comprises a stiffener coupled with a distal portion of the sensor.
Clause 19. A sensor insertion component for use in an applicator of an in vivo analyte sensor, the component comprising:

a sensor module comprising a connector coupled with a sensor; and

a sharp module coupled with the sensor module, the sharp module comprising:
a base;
an elongate protector coupled with the base, the elongate protector comprising a longitudinal axis and a channel configured to receive an intermediate portion of the sensor disposed along a length thereof with a distal portion of the sensor extending past a distal end thereof; and
a sharp coupled with the elongate protector or the base, the sharp comprising a proximal portion and a distal portion, wherein the distal portion extends past a distal end of the elongate protector at an angle to the longitudinal axis of the elongate protector, wherein the angle is between about 5° and about 15°.
Clause 20. The component of clause 19, wherein the distal portion of the sharp is not attached to the elongate protector.
Clause 21. The component of any of the preceding clauses, wherein the proximal portion of the sharp is attached to the elongate protector.
Clause 22. The component of any of the preceding clauses, wherein the sharp further comprises a bent portion between the proximal and distal portion.
Clause 23. The component of clause 22, wherein the bent portion comprises a single deflection having an angle formed by the proximal and distal portions of the sharp, wherein the angle is between about 160° and about 175°.
Clause 24. The component of any of the preceding clauses, wherein the elongate protector has a first side and a second side, wherein the first side comprises the channel, and the proximal portion of the sharp is attached to the second side of the elongate protector.
Clause 25. The component of any of the preceding clauses, wherein the channel of the elongate protector is U-shaped.
Clause 26. The component of any of the preceding clauses, wherein the channel extends along a distal portion of the elongate protector.
Clause 27. The component of any of the preceding clauses, wherein the channel does not extend along a proximal portion of the elongate protector.
Clause 28. The component of any of the preceding clauses, wherein the sharp comprises a solid needle having a diameter not greater than about 0.5 mm.
Clause 29. The component of any of the preceding clauses, wherein the angle is about 7°.
Clause 30. The component of any of the preceding clauses, wherein the distal portion of the sharp has a length in a range of about 1.0 to about 5.0 mm.
Clause 31. The component of any of the preceding clauses, wherein the sensor further comprises a bump having a traction surface disposed for engagement with a distal end of the elongate protector for transmission of an insertion force along an insertion force vector that is substantially parallel to the longitudinal axis of the elongate protector.
Clause 32. The component of clause 31, wherein the bump comprises a sensor chip.
Clause 33. The component of clause 32, wherein the sensor chip is encased in a protective membrane.
Clause 34. The component of any of the preceding clauses, wherein the sensor further comprises a stiffener coupled with a distal portion of the sensor.
Clause 35. A method for inserting a distal portion of an analyte sensor into a subject using an applicator, the method comprising:

positioning an applicator against a skin surface of the subject, the applicator comprising a housing, a sensor module comprising a connector coupled with a proximal end of a sensor, and a sharp module coupled with the sensor module, wherein the sharp module comprises:
a base;
an elongate protector coupled with the base, the elongate protector comprising a longitudinal axis and a channel configured to receive an intermediate portion of the sensor disposed along a length thereof with a distal portion of the sensor extending past a distal end thereof; and
a sharp coupled with the elongate protector or the base, the sharp comprising a proximal portion and a distal portion, wherein the distal portion extends past a distal end of the elongate protector at an angle to the longitudinal axis of the elongate protector, wherein the angle is between about 5° and about 15°;

inserting a distal end of the sharp into a skin of the subject by applying a force against a proximal portion of the housing, wherein the angle of the distal portion causes stretching of skin around a shaft of the needle and creates an opening;

inserting a distal end of the sensor into the opening; and

retracting the sharp.
Clause 36. The method of clause 35, wherein the distal portion of the sharp is not attached to the elongate protector.
Clause 37. The method of any of the preceding clauses, wherein the proximal portion of the sharp is attached to the elongate protector.
Clause 38. The method of any of the preceding clauses, wherein the sharp further comprises a bent portion between the proximal and distal portion.
Clause 39. The method of clause 38, wherein the bent portion comprises a single deflection having an angle formed by the proximal and distal portions of the sharp, wherein the angle is between about 160° and about 175°.
Clause 40. The method of any of the preceding clauses, wherein the elongate protector has a first side and a second side, wherein the first side comprises the channel, and the proximal portion of the sharp is attached to the second side of the elongate protector.
Clause 41. The method of any of the preceding clauses, wherein the channel of the elongate protector is U-shaped.
Clause 42. The method of any of the preceding clauses, wherein the channel extends along a distal portion of the elongate protector.
Clause 43. The method of any of the preceding clauses, wherein the channel does not extend along a proximal portion of the elongate protector.
Clause 44. The method of any of the preceding clauses, wherein the sharp comprises a solid needle having a diameter not greater than about 0.5 mm.
Clause 45. The method of any of the preceding clauses, wherein the angle is about 7°.
Clause 46. The method of any of the preceding clauses, wherein the distal portion of the sharp has a length in a range of about 1.0 to about 5.0 mm.
Clause 47. A sensor insertion component for use in an applicator of an in vivo analyte sensor, the component comprising:

a sensor module holding a connector coupled with a proximal end of a flexible elongate sensor, wherein the sensor module comprises at least one surface defining a skin normal insertion force vector; and

a sharp module held by the sensor module and configured for motion relative to the sensor module parallel to the skin normal insertion force vector, wherein the sharp module comprises:
a base configured for the motion relative to the sensor module;
a U-shaped protector fixed to the base, having an intermediate portion of the flexible elongate sensor disposed along a length thereof with a distal portion of the flexible elongate sensor extending past a distal end thereof; and
a sharp fixed to at least one of the base or the U-shaped protector, the sharp having an outer diameter not greater than 0.56 mm and a distal portion extending past a distal end of the flexible elongate sensor at an angle to the skin normal insertion force vector of not less than five degrees and not greater than fifteen degrees.
Clause 48. The sensor insertion component of clause 47, wherein the sharp comprises a solid needle having a diameter not greater than about 0.5 mm.
Clause 49. The sensor insertion component of clause 48, wherein the diameter of the sharp is less than or equal to about 0.35 mm.
Clause 50. The sensor insertion component of any of the preceding clauses, wherein the motion is a sliding motion.
Clause 51. The sensor insertion component of any of the preceding clauses, wherein the sharp is aligned within about 7° of the skin normal insertion force vector.
Clause 52. The sensor insertion component of any of the preceding clauses, wherein the intermediate portion of the flexible elongate sensor is disposed in a channel of the U-shaped protector.
Clause 53. The sensor insertion component of any of the preceding clauses, wherein the distal portion of the sharp has a length in a range of about 1.0 to about 5.0 mm.
Clause 54. The sensor insertion component of any of the preceding clauses, wherein the distal portion of the flexible elongate sensor has a length in a range of about 0.5 to about 4.0 mm.
Clause 55. The sensor insertion component of any of the preceding clauses, wherein the U-shaped protector has a length extending from the base in the range of about 1.0 to about 10 mm.
Clause 56. The sensor insertion component of any of the preceding clauses, wherein the sharp is fixed to the base.
Clause 57. The sensor insertion component of any of the preceding clauses, wherein the sharp is fixed to the U-shaped protector.
Clause 58. The sensor insertion component of any of the preceding clauses, wherein a distal end of the flexible elongate sensor is sharpened to a point.
Clause 59. The sensor insertion component of any of the preceding clauses, wherein a distal end of the flexible elongate sensor touches a shaft of the sharp.
Clause 60. The sensor insertion component of any of the preceding clauses, wherein a distal end of the flexible elongate sensor is disposed along a shaft of the sharp.
Clause 61. The sensor insertion component of any of the preceding clauses, further comprising a bump having a traction surface attached to the flexible elongate sensor, with the traction surface disposed for engagement with the distal end of the U-shaped protector for transmission of an insertion force along the skin normal insertion force vector to the flexible elongate sensor.
Clause 62. The sensor insertion component of clause 61, wherein the bump comprises a sensor chip.
Clause 63. The sensor insertion component of clause 62, wherein the sensor chip is encased in a protective membrane.
Clause 64. The sensor insertion component of clause 62, wherein the sensor chip is coupled with the connector by a conductor disposed along the flexible elongate sensor.
Clause 65. The sensor insertion component of clause 62, wherein the sensor chip comprises a thermistor.
Clause 66. The sensor insertion component of any of the preceding clauses, further comprising a stiffener coupled with a distal portion of the flexible elongate sensor.
Clause 67. The sensor insertion component of clause 66, wherein the stiffener provides a sensing function.
Clause 68. A method for inserting a distal portion of an analyte sensor into a subject using an applicator, the method comprising:

inserting a needle into a skin of a subject fixed at an angle of about 5 to about 15 degrees to a skin normal insertion force vector, causing stretching of skin around a shaft of the needle;

inserting a tip of a flexible elongate sensor into an opening created by the stretching of the skin to a desired depth and waiting for a delay period; and

retracting the needle after the delay period.
Clause 69. The method of clause 68, wherein inserting the tip of the flexible elongate sensor further comprises supporting an intermediate portion of the flexible elongate sensor using a U-shaped protector during the inserting.
Clause 70. The method of clause 69, further comprising pushing, by a distal end of the U-shaped protector, on a traction surface placed on a distal portion of the flexible elongate sensor.
Clause 71. The method of any of the preceding clauses, performed using the sensor insertion component of clause 47.
Clause 72. The method of clause 71, wherein the delay period is between 0.5 and 3 seconds.
Clause 73. The method of clause 71, wherein the delay period is 1 second.
Clause 74. A needle assembly comprising:

a support material having a plurality of apertures defined therein;

an elongate protector located in each aperture of the plurality of apertures, the elongate protector comprising a U-shaped channel, a longitudinal axis, a proximal end, and a distal end, wherein the distal end is coupled with the support material;

a first injection molded coupler located within each aperture of the plurality of apertures, wherein the first injection molded coupler surrounds the proximal end of the elongate protector and a portion of the support material; and

a sharp located in each aperture of the plurality of apertures, the sharp comprising a proximal portion, a distal portion, and a bent portion between the proximal and distal portion, wherein the proximal portion of the sharp is coupled with the elongate protector and the distal portion of the sharp is not attached to the elongate protector.
Clause 75. The assembly of clause 74, wherein the support material comprises a continuous metal tape.
Clause 76. The assembly of any of the preceding clauses, wherein the bent portion comprises a single deflection.
Clause 77. The assembly of any of the preceding clauses, wherein a neck extends from the support material into each of the plurality of apertures and connects to the elongate protector, and wherein the first injection molded coupler surrounds the neck and a proximal end of the elongate protector.
Clause 78. The assembly of clause 77, wherein the neck has a longitudinal axis and wherein the longitudinal axis of the neck is parallel to the longitudinal axis of the elongate coupler.
Clause 79. The assembly of any of the preceding clauses, further comprising a second injection molded coupler located within each aperture of the plurality of apertures. wherein the second injection molded coupler surrounds a distal end of the sharp.
Clause 80. The assembly of clause 79, wherein the second injection molded coupler connects the sharp to a second portion of the support material.
Clause 81. The assembly of any of the preceding clauses, wherein the first injection molded coupler does not surround a proximal end of the sharp.
Clause 82. The assembly of any of the preceding clauses, wherein the distal portion of the sharp is held at an angle ranging between about 5° and about 15° to the longitudinal axis of the elongate coupler.
Clause 83. The assembly of any of the preceding clauses, wherein the distal portion of the sharp is held non-parallel with respect to the longitudinal axis of the elongate coupler.
Clause 84. A method comprising:

providing a support material comprising a frame comprising a plurality of apertures defined therein, an elongate protector in each of the plurality of apertures, and a neck extending from the frame to the elongate protector in each of the plurality of apertures;

attaching a sharp to each of the elongate protectors, wherein the sharp comprises a proximal portion, a distal portion, and a bent portion between the proximal and distal portion, wherein the proximal portion of the sharp is attached to the elongate protector and the distal portion of the sharp is not attached to the elongate protector; and

injection molding polymeric material to form a first injection molded coupler that surrounds a portion of the neck and a proximal portion of the elongate coupler, wherein a longitudinal axis of the neck is parallel to a longitudinal axis of the elongate coupler, and wherein the distal portion of the sharp extends past a distal end of the elongate protector at an angle to the longitudinal axis of the elongate protector, wherein the angle is between about 5° and about 15°.
Clause 85. The method of clause 84, wherein the bent portion of the sharp comprises a single deflection.
Clause 86. The method of any of the preceding clauses, wherein the support material comprises a continuous metal tape.
Clause 87. The method of any of the preceding clauses, further comprising the step of: injection molding polymeric material to form a second injection molded coupler that surrounds a distal end of the sharp and connects the sharp to a second location on the support material.
Clause 88. The method of clause 87, further comprising the step of:

separating a needle construct from the support material and the second injection molded coupler, the needle construct comprising the sharp, the elongate protector, and the first injection molded coupler; and

incorporating the needle construct into an insertion device for an analyte sensor.
Clause 89. The method of clause 88, wherein separating the needle construct comprises severing the neck adjacent to the first injection molded coupler.

1. An applicator, comprising:
a housing comprising a distal end configured to be placed against a skin surface;
a sensor module comprising a connector coupled with a sensor;
a sharp module coupled with the sensor module, the sharp module comprising:
a base;
an elongate protector coupled with the base; and
a sharp coupled with the elongate protector or the base, wherein a distal portion of the sharp extends past a distal end of the elongate protector at an angle to a longitudinal axis of the elongate protector;
a retraction spring configured to automatically retract the sharp module and the sharp from the skin surface in a proximal direction; and
sensor electronics configured to be advanced from a proximal position in the housing to a distal position.

2. The applicator of claim 1, wherein the sharp comprises a proximal portion and a bent portion between the proximal portion and the distal portion.

3. The applicator of claim 1, wherein the elongate protector comprises a longitudinal axis and a channel configured to receive an intermediate portion of the sensor disposed along a length thereof with a distal portion of the sensor extending past a distal end thereof.

4. The applicator of claim 3, wherein the channel is U-shaped.

5. The applicator of claim 1, wherein the distal portion of the sharp is not attached to the elongate protector.

6. The applicator of claim 2, wherein the proximal portion of the sharp is attached to the elongate protector.

7. The applicator of claim 2, wherein the bent portion comprises a single deflection having an angle formed by the proximal and distal portions of the sharp, wherein the angle is between about 160° and about 175°.

8. The applicator of claim 3, wherein the elongate protector has a first side and a second side, wherein the first side comprises the channel, and the proximal portion of the sharp is attached to the second side of the elongate protector.

9. The applicator of claim 3, wherein the channel of the elongate protector is U-shaped.

10. The applicator of claim 3, wherein the channel extends along a distal portion of the elongate protector.

11. The applicator of claim 1, wherein the distal portion of the sharp has a length in a range of about 1.0 to about 5.0 mm.

12. The applicator of claim 1, wherein the sharp comprises a solid needle having a diameter not greater than about 0.5 mm.

13. The applicator of claim 1, wherein the angle is between about 5° and about 15°.

14. The applicator of claim 1, wherein the applicator is configured to advance the sensor electronics in a distal direction.

15. The applicator of claim 1, wherein the sensor further comprises a bump having a traction surface disposed for engagement with a distal end of the elongate protector for transmission of an insertion force along an insertion force vector that is substantially parallel to the longitudinal axis of the elongate protector.

16. The applicator of claim 15, wherein the bump comprises a sensor chip.

17. The applicator of claim 16, wherein the sensor chip is encased in a protective membrane.

18. The applicator of claim 1, wherein the sensor further comprises a stiffener coupled with a distal portion of the sensor.

19-89. (canceled)
